(19) United States
(12) Patent Application Publication
(10) Pub. No.: US 2008/0207476 A1
(43) Pub. Date: Aug. 28, 2008

(54) PARTICLES CONTAINING PERFUME HAVING IMPROVED FRAGRANCE PROPERTIES

(75) Inventors: Rene-Andres Artiga Gonzalez, Dusseldorf (DE); Andreas Bauer, Kaarst (DE); Robert Stephen Cappleman, Duisburg (DE); Hubert Harth, Hilden (DE); Jurgen Hilsmann, Dusseldorf (DE); Wolfgang Lahn, Willich (DE); Mario Sturm, Leverkusen (DE)

Correspondence Address: Ratner Prestia, Suite 301, 1235 Westlakes Drive, Berwyn, PA, 19312, US

(73) Assignee: Henkel Kommanditgesellschaft Auf Aktien, Dusselforf (DE)

(21) Appl. No.: 12/041,239

(22) Filed: Mar. 3, 2008

Related U.S. Application Data
(63) Continuation of application No. PCT/EP2006/007955, filed on Aug. 11, 2006.

(30) Foreign Application Priority Data
Sep. 2, 2005 (DE) 10 2005 042 054

(51) Int. Cl. A61K 8/11; C11D 3/50; A61Q 13/00

(52) U.S. Cl. 510/101; 512/4

(57) ABSTRACT

A particle containing a carbonate, a sulfate, a perfume, and a layered silicate, the particle having a weight ratio of layered silicate to carbonate and sulfate combined of ≦1:2. The particles, having a desirable fragrance profile, are useful in detergents, fabric softeners, and textile treatments.

### CROSS-REFERENCE TO RELATED APPLICATIONS

This application is a continuation under 35 U.S.C. §§ 120 and 365(c) of international application PCT/EP2006/007955, filed on Aug. 11, 2006. This application also claims priority under 35 U.S.C. § 119 of DE 10 2005 042 054.0, filed on Sep. 2, 2005.

### BACKGROUND OF THE INVENTION

The present invention relates to sulfate-, carbonate-, and perfume-containing particles that contain layered silicate, the ratio of layered silicate to the total quantity of carbonate and sulfate being ≦1:2. It further relates to the use of said particles, to methods for the manufacture thereof, and to agents containing said particles.

In textile washing, treatment, and post-treatment, it is usual today to mix into the washing agents and post-treatment agents certain quantities of perfume that serve to impart a pleasant scent to the washing or rinsing bath itself, but also to the textile items treated with the washing or rinsing bath. The scenting of washing or cleaning agents as well as post-treatment agents is, in addition to color and appearance, an important aspect of the esthetic product impression, and an important factor in the consumer's decision for or against a specific product. For scenting, the perfume either can be incorporated directly into the agent or delivered into the washing or rinsing bath in an additional step. The first approach defines a specific product characteristic; with the second approach, the computer can decide individually as to “his” or “her” scent from a number of scent variants that are offered, comparably to the selection of an eau de toilette or an aftershave.

Scent particles and methods for scenting washing and rinsing baths are known as such from an extensive existing art, carrier materials of an organic or inorganic nature, such as, for example, starches, silicic acids, phosphates, zeolites, alkali salts of polycarboxylic acids, cyclodextrins, etc., usually being brought together with fragrances and further adjuvants.

Correspondingly known fragrance particles either require additional barrier or sheathing layers in order to immobilize the perfume on the carrier, or are not equally suitable for scenting washing or cleaning agents and for direct use as a sole scenting agent, for example for the rinse cycle in a washing machine.

Those fragrance particles that contain zeolite as a carrier material exhibit an additional problem. It has been observed that precisely those fragrance particles that contain larger quantities of zeolite often produce stability problems in terms of the perceptible scent impression, so that, for example, the odor characteristic of a fragrance particle changes in definitely disadvantageous fashion especially after several weeks of storage, so that, for example, an initially pleasant-smelling particle then smells rather foul. It may therefore be assumed that at least individual constituents of a fragrance composition with which a corresponding particle is impregnated fall victim to at least partial decomposition during storage of the particle, with the result that the originally harmonious odor profile is lost.

For this reason, a need exists for further fragrance particles that ensure a similar perfume loading capability but also an improved perfume stability, as compared with what is usual for fragrance particles that contain larger quantities of zeolite. In particular, good scent stability even after several weeks of storage should be possible.

### DESCRIPTION OF THE INVENTION

It has now been found, in unforeseen fashion, that this object is achieved by the subject matter of the present invention, namely a particle containing carbonate(s), sulfate(s), and perfume that is by preference absorbed into the particle and/or adsorbed onto the particle, as well as layered silicates, the ratio of layered silicate to the total quantity of carbonate and sulfate being ≦1:2.

The Applicant has unexpectedly found that corresponding particles that combine carbonate, sulfate, and layered silicate in the corresponding quantitative ratios result in greatly improved scent stability even with several weeks of storage, as compared with particles that contained substantially zeolite as carrier material. Advantageously, the particles according to the present invention also have a very high perfume-oil acceptance capacity that is at least comparable with, if not greater than, that of usual zeolite-based carriers. Advantageously, the particles according to the present invention also have very good powder properties even with high perfume loading, i.e. they are readily pourable and do not clump.

The particles according to the present invention result, advantageously, in a more intensive fragrance experience for the consumer, for example when washing laundry with a detergent formulation that contains the particles according to the present invention. A fragrance-intensifying effect is therefore achieved here, which affects the particles directly as well as objects into which said particles are incorporated, for example detergent formulations, as well as things such as, for example, textiles that are treated with the objects (in this case, a detergent formulation).

A further advantage of the particles according to the present invention is, advantageously, the fact that the scent impression resulting from the particles persists, indirectly and directly, for longer. “Directly” means in this connection that the particles according to the present invention as such are fragrant over a longer period of time. “Indirectly” means in this connection that objects (e.g. a detergent formulation) that contain the particles according to the present invention are fragrant for longer, and that in fact when said objects (e.g. a detergent formulation for washing textiles) are used, the things (in this case, a washed textile) treated therewith are fragrant for longer.

A scent-retarding action (i.e. an extension over time of the scent impression) is therefore achieved, in relation to both the particles and to objects containing the particles, and to the things treated with said objects.

According to a preferred embodiment, the layered silicate to be used refers to
a) double-layered silicates such as, by preference, silicates of the kaolin and/or serpentine group such as, in particular, kaolinite, dickite, halloysite, antigorite, lizardite, and/or chrysotile;
b) triple-layered silicates such as, by preference, pyrophyllite, talc, silicates of the mica group such as, for example, muscovite; of the smectite group such as, in particular, montmorillonite, beidellite, hectorite, nontronite, and/or saponite; of the hydromica group such as, for example, illite, seladonite, and glauconite; as well as vermiculites and chlorites such as, by preference, clinochlore, chamosite, and/or donbassite;
c) layered silicates having mixed layer structures such as, by preference, mixed-layer muscovite-montmorillonite (also mixed-layer illite-smectite);
d) clay minerals having a fiber structure such as, by preference, sepiolith and/or palygorskite; and/or
e) non-crystalline silicates such as, for example, allophane and imogolite.

Layered silicates are found, for example, in clay, in which silicate particles generally predominate. Clay minerals of this kind such as, for example, kaolinite, illite, montmorillonite, etc. are produced principally by the weathering of, for example, feldspars. Bentonites are specific clays that contain smectites, chiefly montmorillonite, as principal minerals; in addition, for example, mica, illite, and cristobalite can be present as contaminants. Bentonites are advantageously usable for the invention, so that according to a preferred embodiment the particles contain layered silicate in the form of clay, by preference smectite-rich clay, in particular bentonite.

It is known that the properties of clays, in particular of bentonite, can be modified. For example, it is possible to increase the swellability of raw bentonite by exchanging the calcium ions for sodium ions (e.g. activated calcium bentonite). It is likewise possible, for example, to increase the specific surface by treatment with inorganic acids (e.g. acid-activated bentonite). It is similarly possible to increase organophily by, for example, reacting sodium bentonite with quaternary ammonium compounds (e.g. organophilic bentonites or bentones). For purposes of this invention, the terms “layered silicates,” “clays,”, “bentonites,” “montmorillonite,” “hectorite,” etc. also encompass the corresponding derivatives or modified substances.

According to a preferred embodiment, the particle contains layered silicates in quantities of more than 1 wt %, by preference more than 3 wt %, advantageously more than 5 wt %, in more greatly advantageous fashion more than 8 wt %, in more advantageous fashion more than 10 wt %, in particular in quantities of at least 15 wt %, based on the entire particle. Layered silicate can also be contained in even greater quantities, for example in quantities of at least 20 wt % or even at least 25 wt %. The minimum limit can also be located at one of the values located therebetween, i.e. for example at a value of 2 wt %, 4 wt %, 6 wt %, 7 wt %, 9 wt %, 11 wt %, 12 wt %, 13 wt %, 14 wt %, 16 wt %, 17 wt %, 18 wt %, 19 wt %, 21 wt %, 22 wt %, 23 wt %, or 24 wt %, based on the entire particle.

In the context of a different embodiment, however, it can also be desirable for the layered silicate content in fact to be limited. According to such an embodiment, the particle contains less than 40 wt %, by preference less than 30 wt %, advantageously less than 25 wt %, in more greatly advantageous fashion less than 20 wt %, in more advantageous fashion less than 15 wt %, in even more advantageous fashion no more than 10 wt % layered silicate, based on the entire particle.

Layered silicate quantities from 3 to 25 wt %, by preference from 5 to 10 wt %, based on the entire particle, can be very advantageous. This corresponds to a preferred embodiment, as do layered silicate quantities according to another possible combination of the aforementioned quantities.

The term “perfume” means perfume oils, fragrances, and odorants. Individual odorant compounds, e.g. the synthetic products of the ester, ether, aldehyde, ketone, alcohol, and hydrocarbon types, can be used as perfume oils or fragrances. Odorant compounds of the ester type are, for example, benzyl acetate, phenoxyethyl isobutyrate, p-tert.-butyl cyclohexyl acetate, linalyl acetate, dimethyl benzyl carbinyl acetate (DMBCA), phenyl ethyl acetate, benzyl acetate, ethyl methyl phenyl glycinate, allyl cyclohexyl propionate, styrallyl propionate, benzyl salicylate, cyclohexyl salicylate, floramate, melusate, and jasmecyclate. The ethers include, for example, benzyl ethyl ether and ambroxan; the aldehydes, for example, the linear alkanals having 8 to 18 carbon atoms, citral, citronellal, citronellyl oxyacetaldehyde, cyclamenaldehyde, lilial and bourgeonal; the ketones, for example, the ionones, α-isomethyl ionone und methyl cedryl ketone; the alcohols, anethol, citronellol, eugenol, geraniol, linalool, phenylethyl alcohol and terpineol; and the hydrocarbons include principally the terpenes such as limonene and pinene. Preferably, however, mixtures of different odorants that together produce an attractive fragrance note are used.

Such perfume oils can also contain natural odorant mixtures such as those accessible from plant sources, for example pine, citrus, jasmine, patchouli, rose, or ylang-ylang oil. Also suitable are muscatel, salvia oil, chamomile oil, clove oil, lemon balm oil, mint oil, cinnamon leaf oil, linden blossom oil, juniper berry oil, vetiver oil, olibanum oil, galbanum oil, and labdanum oil, as well as orange blossom oil, neroli oil, orange peel oil, and sandalwood oil.

In order to be perceptible, an odorant must be volatile; in addition to the nature of the functional groups and the structure of the chemical compound, the molecular weight also plays an important part. Most odorants, for example, possess molar weights of up to approximately 200 dalton, while molar weights of 300 dalton and above represent something of an exception. Because of the differing volatility of odorants, the odor of a perfume or fragrance made up of multiple odorants changes during volatilization, the odor impressions being subdivided into a “top note,” “middle note” or “body,” and “end note” or “dry out.” Because the perception of an odor also depends a great deal on the odor intensity, the top note of a perfume or fragrance is not made up only of highly volatile compounds, while the end note comprises for the most part less-volatile, i.e. adherent odorants.

Adherent odorants that are advantageously usable in the context of the present invention are, for example, the essential oils such as angelica oil, anise oil, arnica flower oil, basil oil, bay oil, bergamot oil, champaca flower oil, silver fir oil, silver fir cone oil, elemi oil, eucalyptus oil, fennel oil, fir needle oil, galbanum oil, geranium oil, gingergrass oil, guaiac wood oil, balsam gurjun oil, helichrysum oil, ho oil, ginger oil, iris oil, cajeput oil, calamus oil, chamomile oil, camphor oil, kanaga oil, cardamom oil, cassia oil, pine needle oil, balsam copaiva oil, coriander oil, curled peppermint oil, caraway oil, cumin oil, lavender oil, lemon grass oil, lime oil, tangerine oil, lemon balm oil, ambrette seed oil, myrrh oil, clove oil, neroli oil, niaouli oil, olibanum oil, orange oil, oregano oil, palmarosa oil, patchouli oil, balsam peru oil, petitgrain oil, pepper oil, peppermint oil, pimento oil, pine oil, rose oil, rosemary oil, sandalwood oil, celery oil, spik oil, star anise oil, turpentine oil, thuja oil, thyme oil, verbena oil, vetiver oil, juniper berry oil, wormwood oil, wintergreen oil, ylang-ylang oil, ysop oil, cinnamon oil, cinnamon leaf oil, citronella oil, lemon oil, and cypress oil.

The higher-boiling or solid odorants of natural or synthetic origin can, however, also be advantageously used in the context of the present invention as adherent odorants or odorant mixtures, i.e. fragrances. These compounds include the compounds recited below, and mixtures thereof: ambrettolide, α-amyl cinnamaldehyde, anethole, anisealdehyde, anise alcohol, anisole, anthranilic acid methyl ester, acetophenone, benzyl acetone, benzaldehyde, benzoic acid ethyl ester, benzophenone, benzyl alcohol, benzyl acetate, benzyl benzoate, benzyl formate, benzyl valerate, bomeol, bornyl acetate, α-bromostyrene, n-decylaldehyde, n-dodecylaldehyde, eugenol, eugenol methyl ether, eucalyptol, farnesol, fenchone, fenchyl acetate, geranyl acetate, geranyl formate, heliotropin, heptyne carboxylic acid methyl ester, heptaldehyde, hydroquinone dimethyl ether, hydroxycinnamaldehyde, hydroxycinnamyl alcohol, indole, irone, isoeugenol, isoeugenol methyl ether, isosafrol, jasmone, camphor, carvacrol, carvone, p-cresol methyl ether, cumarin, p-methoxyacetophenone, methyl n-amyl ketone, methylanthranilic acid methyl ester, p-methyl acetophenone, methylchavicol, p-methyl quinoline, methyl-β-naphthyl ketone, methyl n-nonylacetaldehyde, methyl n-nonyl ketone, muscone, β-naphthol ethyl ether, β-naphthol methyl ether, nerol, nitrobenzene, n-nonylaldehyde, nonyl alcohol, n-octylaldehyde, p-oxyacetophenone, pentadecanolide, β-phenylethyl alcohol, phenylacetaldehyde dimethyl acetal, phenylacetic acid, pulegone, safrole, salicylic acid isoamyl ester, salicylic acid methyl ester, salicylic acid hexyl ester, salicylic acid cyclohexyl ester, santalol, skatole, terpineol, thymene, thymol, γ-undelactone, vanillin, veratrumaldehyde, cinnamaldehyde, cinnamyl alcohol, cinnamic acid, cinnamic acid ethyl ester, cinnamic acid benzyl ester.

Included among the more-volatile odorants advantageously usable in the context of the present invention are, in particular, the lower-boiling odorants of natural or synthetic origin, which can be used alone or in mixtures. Examples of more-volatile odorants are alkyl isothiocyanates (alkylmustard oils), butanedione, limonene, linalool, linalyl acetate and propionate, menthol, menthone, methyl-n-heptenone, phellandrene, phenylacetaldehyde, terpinyl acetate, citral, citronellal.

All the aforesaid odorants are usable alone or in mixed fashion according to the present invention, with the advantages already recited.

The perfume-stabilizing effect according to the present invention relates substantially to the entire fragrance and odorant collective.

The perfume-stabilizing effect also relates in particular to those fragrances and odorants that are or become related to stability problems of perfume on carrier materials, in particular zeolite. Such rather problematic odorants are known to the skilled artisan from daily familiarity.

It is usually the case that such odorants, because of their instability, are not even incorporated into particles in the first place, especially not into zeolite-based ones. The invention here opens up to the skilled artisan a greater freedom in the use of his or her odorants.

Such rather problematic odorants refer in particular to allyl alcohol esters, esters of secondary alcohols, esters of tertiary alcohols, allylic ketones, acetals, ketals, condensation products of amines and aldehydes, and/or mixtures thereof. It is precisely these odorants, especially to the extent they are incorporated as a constituent of a perfume composition into a carrier, in particular a zeolite-based carrier, that result in considerable stability problems and thus disrupt or in fact completely ruin the entire odor profile.

Allyl alcohol esters are the esters of allyl alcohol that exhibit the following structural feature: C(OH)—C═C. Examples of allyl alcohol esters are, in particular, allyl amyl glycolate, allyl anthranilate, allyl benzoate, allyl butyrate, allyl caprate, allyl caproate, allyl cinnamate, allyl cyclohexane acetate, allyl cyclohexane butyrate, allyl cyclohexane propionate, allyl heptoate, allyl nonanoate, allyl salicylate, amyl cinnamyl acetate, amyl cinnamyl formate, cinnamyl formate, cinnamyl acetate, cyclogalbanate, geranyl acetate, geranyl acetoacetate, geranyl benzoate, geranyl cinnamate, methallyl butyrate, methallyl caproate, neryl acetate, neryl butyrate, amyl cinnamyl formate, alpha-methyl cinnamyl acetate, methyl geranyl tiglate, mertenyl acetate, farnesyl acetate, fenchyl acetate, geranyl anthranilate, geranyl butyrate, geranyl isobutyrate, geranyl caproate, geranyl caprylate, geranyl ethyl carbonate, geranyl formate, geranyl furoate, geranyl heptoate, geranyl methoxyacetate, geranyl pelargonate, geranyl phenyl acetate, geranyl phthalate, geranyl propionate, geranyl isopropoxyacetate, geranyl valerate, geranyl isovalerate, trans-2-hexenyl acetate, trans-2-hexenyl butyrate, trans-2-hexenyl caproate, trans-2-hexenyl phenyl acetate, trans-2-hexenyl propionate, trans-2-hexenyl tiglate, trans-2-hexenyl valerate, beta-pentenyl acetate, alpha-phenyl allyl acetate, prenyl acetate, trichloromethyl phenyl carbinyl acetate, and/or mixtures thereof. Allyl alcohol esters can lead to stability problems in carrier materials, in particular with zeolite-based carriers, which have a negative effect on the odor profile of the entire perfume composition. The agents according to the present invention allow perfume compositions that encompass allyl alcohol esters to be better stabilized. The aforesaid odorants can by preference be contained in the particles according to the present invention.

Examples of esters of secondary alcohols (secondary alcohols exist when two hydrogen atoms are substituted with organic radicals (R¹ and R²) on the carbon atom that carries the OH group [general formulas: R¹—CH(OH)—R²]) are, in particular, ortho-tert.-amyl cyclohexyl acetate, isoamyl benzyl acetate, secondary n-amyl butyrate, amyl vinyl carbinyl acetate, amyl vinyl carbinyl propionate, cyclohexyl salicylate, dihydronorcyclopentadienyl acetate, dihydro-norcyclopentadienyl propionate, isobornyl acetate, isobornyl salicylate, isobornyl valerate, frutene, 2-methyl buten-2-ol 4-acetate, methyl phenyl carbinyl acetate, 2-methyl-3-phenyl propan-2-yl acetate, prenyl acetate, 4-tert-butyl cyclohexyl acetate, verdox (2-tert-butyl cyclohexyl acetate), i vertenex (4-tert-butyl cyclohexyl acetate), violiff (carboxylic acid 4-cycloocten-1-yl methyl ester), ethenyl isoamyl carbinyl acetate, fenchyl acetate, fenchyl benzoate, fenchyl-n-butyrate, fenchyl isobutyrate, laevomenthyl acetate, dimenthyl acetate, menthyl anthranilate, menthyl benzoate, menthyl isobutyrate, menthyl formate, laevomenthyl phenyl acetate, menthyl propionate, menthyl salicylate, menthyl isovalerate, cyclohexyl acetate, cyclohexyl anthranilate, cyclohexyl benzoate, cyclohexyl butyrate, cyclohexyl isobutyrate, cyclohexyl caproate, cyclohexyl cinnamate, cyclohexyl formate, cyclohexyl heptoate, cyclohexyl oxalate, cyclohexyl pelargonate, cyclohexyl phenyl acetate, cyclohexyl propionate, cyclohexyl thioglycolate, cyclohexyl valerate, cyclohexyl isovalerate, methyl amyl acetate, methyl benzyl carbinyl acetate, methyl butyl cyclohexanyl acetate, 5-methyl-3-butyl-tetrahydropyran-4-yl acetate, methyl eitrate, methyl isocampholate, 2-methyl cyclohexyl acetate, 4-methyl cyclohexyl acetate, 4-methyl-cyclohexyl methyl carbinyl acetate, methyl ethyl benzyl carbinyl acetate, 2-methyl heptanol-6 acetate, methyl heptenyl acetate, alpha methyl-n-hexyl carbinyl formate, methyl-2-methyl butyrate, methyl nonyl carbinyl acetate, methyl phenyl carbinyl acetate, methyl phenyl carbinyl anthranilate, methyl phenyl carbinyl benzoate, methyl phenyl carbinyl n-butyrate, methylphenyl carbinyl isobutyrate, methyl phenyl carbinyl caproate, methyl phenyl carbinyl caprylate, methyl phenyl carbinyl cinnamate, methyl phenyl carbinyl formate, methyl phenyl carbinyl phenyl acetate, methyl phenyl carbinyl propionate, methyl phenyl carbinyl salicylate, methyl phenyl carbinyl isovalerate, 3-nonanyl acetate, 3-nonenyl acetate, nonanediol-2,3 acetate, nonynol acetate, 2-octanyl acetate, 3-octanyl acetate, n-octyl acetate, sec.-octyl isobutyrate, beta-pentenyl acetate, alpha-phenyl allyl acetate, phenyl ethyl methyl carbinyl isovalerate, phenyl ethylene glycol diphenyl acetate, phenyl ethyethyl carbinyl acetate, phenyl glycol diacetate, sec.-phenyl glycol monoacetate, phenyl glycol monobenzoate, isopropyl caprate, isopropyl caproate, isopropyl caprylate, isopropyl cinnamate, para-isopropyl cyclohexanyl acetate, propyl glycol diacetate, propylene glycol diisobutyrate, propylene glycol dipropionate, isopropyl n-heptoate, isopropyl-n-hept-1-yne carbonate, isopropyl pelargonate, isopropyl propionate, isopropyl undecylenate, isopropyl n-valerate, isopropyl isovalerate, isopropyl sebacinate, isopulegyl acetate, isopulegyl acetoacetate, isopulegyl isobutyrate, isopulegyl formate, thymyl propionate, alpha-2,4-trimethyl cyclohexane methyl acetate, trimethyl cyclohexyl acetate, vanillin triacetate, vanillylidene diacetate, vanillyl vanillate, and/or mixtures thereof. These esters can lead to stability problems in carrier materials, in particular with zeolite-based carriers, which have a negative effect on the odor profile of the entire perfume composition. The agents according to the present invention allow perfume compositions that encompass esters of secondary alcohols to be better stabilized. The aforesaid odorants can by preference be contained in the particles according to the present invention.

Preferred examples of esters of tertiary alcohols (tertiary alcohols are those in which three hydrogen atoms are substituted by organic radicals R¹, R², R³ on the carbon atom that carries the OH group [general formula: R¹R²R³C—OH]) are tertiary amyl acetate, caryophyllene acetate, cedrenyl acetate, cedryl acetate, dihydromyrcenyl acetate, dihydroterpinyl acetate, dimethyl benzyl carbinyl acetate, dimethyl benzyl carbinyl isobutyrate, dimethyl heptenyl acetate, dimethyl heptenyl formate, dimethyl heptenyl propionate, dimethyl heptenyl isobutyrate, dimethyl phenyl ethyl carbinyl acetate, dimethyl phenyl ethyl carbinyl isobutyrate, dimethyl phenyl ethyl carbinyl isovalerate, dihydro-nordicyclopentadienyl acetate, dimethyl benzyl carbinyl butyrate, dimethyl benzyl carbinyl formate, dimethyl benzyl carbinyl propionate, dimethyl phenyl ethyl carbinyl n-butyrate, dimethyl phenyl ethyl carbinyl formate, dimethyl phenyl ethyl carbinyl propionate, elemyl acetate, ethinyl cyclohexyl acetate, eudesmyl acetate, eugenyl cinnamate, eugenyl formate, isoeugenyl formate, eugenyl phenyl acetate, isoeudehyl phenyl acetate, guaiyl acetate, hydroxycitronellyl ethyl carbonate, linallyl acetate, linallyl anthranilate, linallyl benzoate, linallyl butyrate, linallyl isobutyrate, linallyl caproate, linallyl caprylate, linallyl cinnamate, linallyl citronellate, linallyl formate, linallyl heptoate, linallyl n-methyl anthranilate, linallyl methyl tiglate, linallyl pelargonate, linallyl phenyl acetate, linallyl propionate, linallyl pyruvate, linallyl salicylate, linallyl n-valerate, linallyl isovalerate, methyl cyclopentenolone butyrate, methyl cyclopentenolone propionate, methyl ethyl phenyl carbinyl acetate, methyl heptyne carbonate, methyl nicotinate, myrcenyl acetate, myrcenyl formate, myrcenyl propionate, cis-ocimenyl acetate, phenyl salicylate, terpinyl acetate, terpinyl anthranilate, terpinyl benzoate, terpinyl n-butyrate, terpinyl isobutyrate, terpinyl cinnamate, terpinyl formate, terpinyl phenyl acetate, terpinyl propionate, terpinyl n-valerate, terpinyl isovalerate, tributyl acetyl citrate, and/or mixtures thereof. These esters can lead to stability problems in carrier materials, in particular with zeolite-based carriers, which have a negative effect on the odor profile of the entire perfume composition. The agents according to the present invention allow perfume compositions that encompass esters of tertiary alcohols to be better stabilized The aforesaid odorants can by preference be contained in the particles according to the present invention.

Certain esters having just such stability problems can be esters of allylic and secondary or allylic and tertiary alcohols, such as, in particular, amyl vinyl carbinyl acetate, amyl vinyl carbinyl propionate, hexyl vinyl carbinyl acetate, 3-nonenyl acetate, 4-hydroxy-2-hexenyl acetate, linallyl anthranilate, linallyl benzoate, linallyl butyrate, linallyl isobutyrate, linallyl caproate, linallyl caprylate, linallyl cinnamate, linallyl citronellate, linallyl formate, linallyl heptoate, linallyl n-methyl anthranilate, linallyl methyl tiglate, linallyl pelargonate, linallyl phenyl acetate, linallyl propionate, linallyl pyruvate, linallyl salicylate, linallyl n-valerate, linallyl isovalerate, myrtenyl acetate, nerolidyl acetate, nerolidyl butyrate, beta-pentenyl acetate, alpha-phenyl allyl acetate, and/or mixtures thereof. These esters, too, can lead to stability problems in carrier materials, in particular with zeolite-based carriers, which have a negative effect on the odor profile of the entire perfume composition. The agents according to the present invention allow perfume compositions that encompass these esters to be better stabilized. The aforesaid odorants can by preference be contained in the particles according to the present invention.

Allylic ketones are characterized by the following structural feature: C—C(═O)—C═C. Preferred examples are acetyl furan, allethrolone, allyl ionone, allyl pulegone, amyl cyclopentenone, benzylidene acetone, benzylidene acetophenone, alpha-isomethyl ionone, 4-(2,6,6-trimethyl-1-cyclohexen-1-yl) 3-buten-2-one, beta-damascone (1-(2,6,6-trimethylcyclohexen-1-yl)-2-buten-1-one), damascenone (1-(2,6,6-trimethyl-1,3-cyclohexadien-1-yl)-2-buten-1-one), delta-damascone (1-(2,6,6-trimethyl-3-cyclohexen-1-yl)-2-buten-1-one), alpha-ionone (4-(2,6,6-trimethyl-1-cyclohexenyl-1-yl)-3-buten-2-one), beta-ionone (4-(2,6,6-trimethyl-1-cyclohexen-1-yl)-3-buten-2-one), gamma-methyl ionone, (4-(2,6,6-trimethyl-2-cyclohexyl-1-yl)-3-methyl-3-buten-2-one), pulegone, and/or mixtures thereof. Allylic ketones can lead to stability problems in carrier materials, in particular with zeolite-based carriers, which have a negative effect on the odor profile of the entire perfume composition. The agents according to the present invention allow perfume compositions that encompass allylic ketones to be better stabilized. The aforesaid odorants can by preference be contained in the particles according to the present invention.

Acetals are geminal diethers of the general formula R¹CH(OR²)(OR³). Preferred examples are acetaldehyde benzyl beta-methoxyethyl acetal, acetaldehyde diisoamyl acetal, acetaldehyde dipentanediol acetal, acetaldehyde di-n-propyl acetal, acetaldehyde ethyl-trans-3-hexenyl acetal, acetaldehyde phenyl ethylene glycol acetal, acetaldehyde phenyl ethyl n-propyl acetal, cinnamic aldehyde dimethyl acetal, acetaldehyde benzyl beta-methoxyethyl acetal, acetaldehyde diisoamyl acetal, acetaldehyde diethyl acetal, acetaldehyde di-cis-3-hexenyl acetal, acetaldehyde dipentanediol acetal, acetaldehyde di-n-propyl acetal, acetaldehyde ethyl trans-3-hexenyl acetal, acetaldehyde phenyl ethylene glycol acetal, acetaldehyde phenyl ethyl n-propylacetal, acetyl vanillin dimethyl acetal, alpha-amyl cinnamic aldehyde diisopropyl acetal, p-tert.-amyl phenoxyacetaldehyde diethyl acetal, anisaldehyde diethyl acetal, anisaldehyde dimethyl acetal, isoapiole, benzaldehyde diethyl acetal, benzaldehyde di-(ethylene glycol monobutyl ether) acetal, benzaldehyde dimethyl acetal, benzaldehyde ethylene glycol acetal, benzaldehyde glyceryl acetal, benzaldehyde propylene glycol acetal, cinnamic aldehyde diethyl acetal, citral diethyl acetal, citral dimethyl acetal, citral propylene glycol acetal, alpha-methyl cinnamic aldehyde diethyl acetal, alpha-cinnamic aldehyde dimethyl acetal, phenyl acetaldehyde 2,3-butylene glycol acetal, phenyl acetaldehyde citronellyl methyl acetal, phenyl acetaldehyde diallyl acetal, phenyl acetaldehyde diamyl acetal, phenyl acetaldehyde dibenzyl acetal, phenyl acetaldehyde dibutyl acetal, phenyl acetaldehyde diethyl acetal, phenyl acetaldehyde digeranyl acetal, phenyl acetaldehyde dimethyl acetal, phenyl acetaldehyde ethylene glycol acetal, phenyl acetaldehyde glyceryl acetal, citronellal cyclomonoglycol acetal, citronellal diethyl acetal, citronellal dimethyl acetal, citronellal diphenyl ethyl acetal, geranoxyacetaldehyde di ethyl acetal, and/or mixtures thereof.

Acetals can lead to stability problems in carrier materials, in particular with zeolite-based carriers, which have a negative effect on the odor profile of the entire perfume composition. The agents according to the present invention allow perfume compositions that encompass acetals to be better stabilized The aforesaid odorants can by preference be contained in the particles according to the present invention.

Ketals are geminal diethers of the general formula R¹R²C(OR³)(OR⁴). Preferred examples are acetone diethyl ketal, acetone dimethyl ketal, acetophenone diethyl ketal, methyl amyl catechol ketal, methyl butyl catechol ketal, and/or mixtures thereof. Ketals can lead to stability problems in carrier materials, in particular with zeolite-based carriers, which have a negative effect on the odor profile of the entire perfume composition. The agents according to the present invention allow perfume compositions that encompass ketals to be better stabilized. The aforesaid odorants can by preference be contained in the particles according to the present invention.

Preferred examples of condensation products of amines and aldehydes are anisaldehyde methyl anthranilate, aurantiol (hydroxycitronellal methyl anthranilate), verdantiol (4-tert-butyl alpha-methyl dihydrocinnamaldehyde methyl anthranilate), vertosin (2,4-dimethyl-3-cyclohexene carbaldehyde), hydroxycitronellal ethyl anthranilate, hydroxycitronellal linallyl anthranilate, methyl-N-(4-(4-hydroxy-4-methyl pentyl)-3-cyclohexenyl methylidene) anthranilate, methyl naphthyl ketone methyl anthranilate, methyl nonyl acetaldehyde methyl anthranilate, methyl-N-(3,5,5-trimethyl hexylidene) anthranilate, vanillin methyl anthranilate, and/or mixtures thereof. Condensation products of amines and aldehydes can lead to stability problems in carrier materials, in particular with zeolite-based carriers, which have a negative effect on the odor profile of the entire perfume composition. The agents according to the present invention allow perfume compositions that encompass condensation products of amines and aldehydes to be better stabilized The aforesaid odorants can by preference be contained in the particles according to the present invention.

Advantageously, however, not only can perfume stability be improved in connection with those odorants that, in carriers (especially zeolite-based carriers) exhibit a clear predisposition to instability or are considered rather unstable (risk group), such as, for example allyl amyl glycolate and cyclogalbanate, but perfume stability can also be even further improved in connection with other odorants. Such other odorants are, without in the least intending to limit the following listing thereto, for example, Adoxal (2,6,10-trimethyl-9-undecen-1-al), amyl acetate, anisaldehyde (4-methoxybenzaldehyde), Bacdanol (2-ethyl-4-(2,2,3-trimethyl-3-cyclopenten-1-yl)-2-buten-1-ol), benzaldehyde, benzophenone, benzyl acetate, benzyl salicylate, 3-hexen-1-ol, Cetalox (dodecahydro-3A,6,6,9A-tetramethylnaphtho[2,1B]furan), cis-3-hexenyl acetate, cis-3-hexenyl salicylate, citronellol, coumarin, cyclohexyl salicylate, Cymal (2-methyl-3-(para-isopropylphenyl)propionaldehyde), decylaldehyde, ethyl vanillin, ethyl-2-methyl butyrate, ethylene brassylate, eucalyptol, eugenol, Exaltolide (cyclopentadecanolide), Florhydral (3-(3-isopropylphenyl) butanal), Galaxolide (1,3,4,6,7,8-hexahydro-4,6,6,7,8,8-hexamethyl cyclopenta-gamma-2-benzopyran), gamma-decalactone, gamma-dodecalactone, geraniol, geranyl nitrile, Helional (alpha-methyl-3,4, (methylenedioxy)hydrocinnamaldehyde), heliotropin, hexyl acetate, hexyl cinnamaldehyde, hexyl salicylate, Hydroxyambran (2-cyclododecyl-propanol), hydroxycitronellal, Iso E Super (7-acetyl-1,2,3,4,5,6,7,8-octahydro-1,1,6,7-tetramethylnaphthalene), isoeugenol, isojasmone, Koavone (acetyl diisoamylene), laurylaldehyde, IRG 201 (2,4-dihydroxy-3,6-dimethyl benzoic acid methyl ester), Lyral (4-(4-hydroxy-4-methylpentyl)-3-cyclohexene-1-carboxaldehyde), Majantol (2,2-dimethyl-3-(3-methylphenyl)-propanol), Mayor (4-(1-methyl ethyl)cyclohexane methanol), methyl anthranilate, methyl beta-naphthyl ketone, methyl cedrylone (methyl cedrenyl ketone), methyl chavicol (1-methyl-oxy-4,2-propen-1-yl benzene), methyl dihydrojasmonate, methyl nonyl acetaldehyde, musk indanone (4-acetyl-6-tert. butyl-1,1-dimethyl indane), nerol, nonalactone (4-hydroxynonanonic acid, lactone), Norlimbanol (1-(2,2,6-trimethylcyclohexyl)-3-hexanol), p-t-Bucinal (2-methyl-3(para-tert.-butyl phenyl) propionaldehyde), para-hydroxyphenyl butanone, patchouli, phenylacetaldehyde, phenyl ethyl acetate, phenyl ethyl alcohol, phenyl ethyl phenyl acetate, phenyl hexanol/phenoxanol (3-methyl-5-phenyl pentanol), Polysantol (3,3-dimethyl-5-(2,2,3-trimethyl-3-cyclopenten-1-yl)-4-penten-2-ol), Rosaphen (2-methyl-5-phenyl pentanol), sandalwood, alpha-terpinene, Tonalid/Musk Plus (7-acetyl-1,1,3,4,4,6-hexamethyl tetralin), undecalactone, Undecavertol (4-methyl-3-decen-5-ol), undecylaldehyde, undecenaldehyde, vanillin, and/or mixtures thereof. The aforesaid odorants can by preference be contained in the particles according to the present invention.

When the perfume, which by preference is absorbed/adsorbed into/onto the particle, contains at least four, advantageously at least five, in more greatly advantageous fashion at least six, in even more greatly advantageous fashion at least seven, in even more advantageous fashion at least eight, by preference at least nine, in particular at least 10 different odorants, a preferred embodiment of the invention then exists.

Odorants very particularly preferred in the context of this invention, which can be used with advantage, are in particular dihydromyrcenol, 4-tert.-butyl cyclohexyl acetate, tetrahydrolinalool, methyl palmitate, methyl myristate, methyl oleate, 6-methyl gamma-ionone, isobornyl acetate, tonalid, and/or dihydromethyl jasmonate, but in particular dihydromyrcenol and/or 4-tert.-butyl cyclohexyl acetate. Preferred particles can consequently, according to a preferred embodiment, encompass at least one of the foresaid odorants.

If desired, the perfume can also be combined with a perfume fixative. It is assumed that perfume fixatives can delay the evaporation of the more-volatile components of perfumes.

According to a further preferred embodiment, the perfume that is absorbed/adsorbed into/onto the carrier material encompasses a perfume fixative, by preference in the form of diethyl phthalates, musk (derivatives), and mixtures thereof, the quantity of fixative being by preference 1 to 55 wt %, advantageously 2 to 50 wt %, even more advantageously 10 to 45 wt %, in particular 20 to 40 wt % of the entire quantity of perfume.

According to a further preferred embodiment, the particles contain an agent elevating the viscosity of liquids, in particular of perfume, by preference PEG (polyethylene glycol), advantageously having a molecular weight from 400 to 2000, the viscosity-elevating agent being contained in preferred fashion in quantities from 0.1 to 20 wt %, advantageously from 0.15 to 10 wt %, in more greatly advantageous fashion from 0.2 to 5 wt %, in particular from 0.25 to 3 wt %, based on the entire particle.

It has been found that the viscosity of agents elevating the viscosity of liquids, in particular of perfume, make a further contribution to stabilizing the perfume in the particle, in particular when nonionic surfactant is simultaneously present.

The viscosity-elevating agents are by preference polyethylene glycols (abbrev.: PEG) that can be described by the following general formula:

H—(O—CH₂—CH₂)ₙ—OH

in which the degree of polymerization n can vary from approx. 5 to more than 100,000, corresponding to molar weights from 200 to 5,000,000 gmol⁻¹. The products having molar weights below 25,000 gmol⁻¹ are referred to as actual polyethylene glycols, while higher-molecular-weight products are often referred to in the literature as polyethylene oxides (abbrev.: PEOX). The polyethylene glycols used by preference can have a linear or branched structure, linear polyethylene glycols being particularly preferred, and can be end-capped.

Among the particularly preferred polyethylene glycols are those having relative molecular weights between 400 and 2000. It is also possible, in particular, to use polyethylene glycols that are present per se in a liquid state at room temperature and a pressure of 1 bar; chiefly relevant here is polyethylene glycol having a relative molecular weight of 200, 400, and 600.

As has already been mentioned, the structure of a perfume composition is divided into a “top note,” “middle note” or “body,” and an “end note” or “dry out.” The top note (“tete,” “Spitze,” initial odor) substantially encompasses readily volatile odorants by preference of a mostly fresh nature. The middle note (“bouquet,” “corps,” “coeur,” “Herznote,” body) substantially encompasses moderately volatile odorants, by preference usually of a flowery nature, and the end note (“fond,” after-odor) substantially encompasses low-volatility odorants which substantially determine the basic character (principal odor) of the perfume.

This therefore means that the top note substantially determines the first phase of the scent sequence of a perfume or of an agent scented with a perfume, such as, for example, a washing agent. It plays the decisive role in the first impression of the odor experience, i.e. for example when the washing-agent package is opened and when the washing agent is added to the washing machine. The top note should substantially evoke attention and interest in the perfume and thus in the agent scented therewith; for this reason, it represents substantially a mixture of light, volatile substances, although middle and end notes can also in some cases already play a role in the first scent phase. Typical constituents of the top note are, for example, the citrus oils, fruit notes, lavender, dihydromyrcenol, or rose oxide. The skilled artisan is aware of a plurality of further constituents from daily familiarity, or can derive them from the relevant technical literature.

The second, middle phase of the scent sequence of a perfume or of an agent such as, for example, a washing agent scented with the perfume, is determined by the middle note. This is by preference constituted by a mixture of rounder, more complex notes, that give a perfume fullness, character, and a certain direction. It can be distinguished, for example, principally by flowery components such as lily-of-the-valley, jasmine, or rose. In addition, many of the spicy constituents of a perfume such as, for example, eugenol (essential odorant of cloves) can be present here. The skilled artisan is aware of a plurality of further constituents from daily familiarity, or can derive them from the relevant technical literature.

The end note of a perfume (with which, for example, a washing agent is scented) determines the character of the scent. It adheres for a very long time to the scented objects and is composed substantially from heavier, warmer notes. For example, a fine-wood base can be combined with isolated odor carriers of other woods and, for example, also with musk odorants and/or an animal complex as well as typical end notes such as patchouli and vanilla.

Perfume compositions are generally created on the basis of this generally accepted concept of perfume notes: a perfume of complex structure can in fact be made up of several hundred individual components. Experience indicates that often only a very well-balanced mixture of many constituents (for example, at least 15 or 10, in many cases at least 30 or 50, or even more) results in perfume-technology success, i.e. a pleasant odor.

Against this background, the significance of the present invention becomes even more evident. On the one hand, it is clear that the degeneration (i.e. breakdown or decomposition) of even a single odorant is substantially sufficient to ruin the harmonious overall structure of an entire perfume composition. At the same time, exactly one single odorant can be necessary in order to ensure the perfume-technology success of a perfume composition. An olfactory diminution in only one note, i.e. the top note, middle, or end note, is already sufficient to considerably diminish the olfactory value of the entire perfume composition, or even entirely wipe it out. Against this background, the successful perfuming of economical mass-market goods such as, for example, washing or cleaning agents, is an undertaking that requires much experience and outstandingly trained personnel. Perfume compositions must, in this context, be adapted to a wide variety of, in some cases, aggressive media and substrates. In the field of washing agents, for example, their alkalinity is highly problematic for many perfume compositions, as is the use of zeolite-containing carrier materials. The present invention now opens up to the perfumer an entirely new spectrum of perfume-technology capabilities for the scenting of washing or cleaning agents using separate fragrance carriers. The skilled artisan can now use even those odorants that earlier, because of their instability especially in zeolite-containing carriers, could not have been considered. He or she can now configure the top note, middle note, and/or end note of a perfume composition even more freely, and balance them more individually. Odorants that would otherwise be rather unstable can now also be given quantitatively greater weight in the perfume compositions.

According to a preferred embodiment, the notes of the perfume composition contained in the particle according to the present invention differ in terms of their quantitative weighting, such that by preference
(a) the top note is weighted quantitatively more highly than the middle note and end note, such that the two lower-weighted notes can be weighted substantially equally to one another, or one of the lower-weighted notes is weighted more highly than the other; or
(b) the middle note is weighted quantitatively more highly than the top note and end note, such that the two lower-weighted notes can be weighted substantially equally to one another, or one of the lower-weighted notes is weighted more highly than the other; or
(c) the end note is weighted quantitatively more highly than the top note and middle note, such that the two lower-weighted notes can be weighted substantially equally to one another, or one of the lower-weighted notes is weighted more highly than the other.

The fact that one note is weighted quantitatively more highly than another means that the total mass of the odorants constituting the higher-weighted note is greater than the total mass of the odorants constituting the lower-weighted note, advantageously by at least 10 wt %, by preference at least 20 wt %, in particular at least 30 wt %, based on the total mass of the entire perfume composition.

According to another preferred embodiment, all the notes of the perfume composition are weighted substantially equally.

As has already been emphasized, the present invention allows the skilled artisan an expanded freedom of action in the scenting of particles, giving him or her the capability of producing particles having additionally refined fragrance notes. For this purpose, according to a preferred embodiment the particle according to the present invention can also contain, in particular, odorants having
(a) an almond-like odor, such as by preference benzaldehyde, pentanal, heptenal, 5-methyl furfural, methyl butanal, furfural, and/or acetophenone, or
(b) an apple-like odor, such as by preference (S)-(+)-ethyl-2-methyl butanoate, diethyl malonate, ethyl butyrate, geranyl butyrate, geranyl isopentanoate, isobutyl acetate, linalyl isopentanoate, (E)-β-damascone, heptyl-2-methyl butyrate, methyl-3-methyl butanoate, 2-hexenal pentyl methyl butyrate, ethyl methyl butyrate, and/or methyl-2-methyl butanoate, or
(c) an apple-skin-like odor, such as by preference ethyl hexanoate, hexyl butanoate, and/or hexyl hexanoate, or
(d) an apricot-like odor, such as by preference γ-undecalactone, or
(e) a banana-like odor, such as by preference isobutyl acetate, isoamyl acetate, hexenyl acetate, and/or pentyl butanoate, or
(f) a bitter-almond-like odor, such as by preference 4-acetyl toluene, or
(g) a blackcurrant-like odor, such as by preference mercaptomethylpentanone and/or methoxymethylbutanethiol, or
(h) a citrusy odor, such as by preference linalyl pentanoate, heptanal, linalyl isopentanoate, dodecanal, linalyl formate, α-p-dimethylstyrene, p-cymenol, nonanal, β-cubebene, (Z)-limonene oxide, cis-6-ethenyl tetrahydro-2,2,6-trimethylpyran-3-ol, cis-pyranoid linalool oxide, dihydrolinalool, 6(10)-dihydromyrcenol, dihydromyrcenol, β-farnesene, (Z)-β-farnesene, (Z)-ocimene, (E)-limonene oxide, dihydroterpinyl acetate, (+)-limonene, (epoxymethylbutyl) methyl furan, and/or p-cymene, or
(i) a cocoa-like odor, such as by preference dimethylpyrazine, butyl methyl butyrate, and/or methyl butanal, or
(j) a coconut-like odor, such as by preference γ-octalactone, γ-nonalactone, methyl laurate, tetradecanol, methyl nonanoate, (3S,3aS,7aR)-3a,4,5,7a-tetrahydro-3,6-dimethylbenzofuran-2(3H)-one, 5-butyldihydro-4-methyl-2(3H)-furanone, ethyl undecanoate, and/or δ-decalactone, or
(k) a cream-like odor, such as by preference diethyl acetal, 3-hydroxy-2-butanone, 2,3-pentadione, and/or 4-heptenal, or
(l) a flowery odor, such as by preference benzyl alcohol, phenylacetic acid, tridecanal, p-anisyl alcohol, hexanol, (E,E)-farnesyl acetone, methyl geranate, trans-crotonaldehyde, tetradecylaldehyde, methyl anthranilate, linalool oxide, epoxylinalool, phytol, 10-epi-γ-eudesmol, nerol oxide, ethyl dihydrocinnamate, γ-dodecalactone, hexadecanol, 4-mercapto-4-methyl-2-pentanol, (Z)-ocimene, cetyl alcohol, nerolidol, ethyl-(E)-cinnamate, elemicin, pinocarveol, α-bisabolol, (2R,4R)-tetrahydro-4-methyl-2-(2-methyl-1-propenyl)-2H-pyran, (E)-isoelemicin, methyl-2-methyl propanoate, trimethylphenylbutenone, 2-methyl anisole, β-farnesol, (E)-isoeugenol, nitrophenylethane, ethyl vanillate, 6-methoxyeugenol, linalool, β-ionone, ethyl benzoate, phenyl ethyl benzoate, isoeugenol, and/or acetophenone, or
(m) a fresh odor, such as by preference methyl hexanoate, undecanone, (Z)-iimonene oxide, benzyl acetate, ethyl hydroxyhexanoate, isopropyl hexanoate, pentadecanal, β-elemene, α-zingiberene, (E)-limonene oxide, (E)-p-mentha-2,8-dien-1-ol, menthone, piperitone, (E)-3-hexenol, and/or carveol, or
(n) a fruit odor, such as by preference ethyl phenyl acetate, geranyl valerate, γ-heptalactone, ethyl propionate, diethyl acetal, geranyl butyrate, ethyl heptylate, ethyl octanoate, methyl hexanoate, dimethyl heptenal, pentanone, ethyl 3-methyl butanoate, geranyl isovalerate, isobutyl acetate, ethoxypropanol, methyl 2-butenal, methyl nonanedione, linalyl acetate, methyl geranate, limonene oxide, hydrocinnamic alcohol, diethyl succinate, ethyl hexanoate, ethyl methylpyrazine, β-cubebene, nryletate, citronellyl butyrate, hexyl acetate, nonyl acetate, butyl methyl butyrate, pentenal, isopentyl dimethylpyrazine, p-menth-1-en-9-ol, hexadecanone, octyl acetate, γ-dodecalactone, epoxy-β-ionone, ethyl octenoate, ethyl isohexanoate, isobornyl propionate, cedrenol, p-menth-1-en-9-yl acetate, cadinadiene, (Z)-3-hexenyl hexanoate, ethyl cyclohexanoate, 4-methylthio-2-butanone, 3,5-octadienone, methyl cyclohexane carboxylate, 2-pentylthiophene, α-ocimene, butanediol, ethyl valerate, pentanol, isopiperitone, butyl octanoate, ethyl vanillate, methyl butanoate, 2-methyl butyl acetate, propyl hexanoate, butyl hexanoate, isopropyl butanoate, spathulenol, butanol, δ-dodecalactone, methyl quinoxaline, sesquiphellandrene, 2-hexenol, ethyl benzoate, isopropyl benzoate, ethyl lactate, and/or citronellyl isobutyrate, or
(o) a geranium-like odor, such as by preference geraniol, (E,Z)-2,4-nonadienal, octadienone, and/or o-xylene, or
(p) a grape-like odor, such as by preference ethyl decanoate and/or hexanone, or
(q) a grapefruit-like odor, such as by preference (+)-5,6-dimethyl-8-isopropenyl bicyclo[4.4.0]dec-1-en-3-one and/or p-menthenethiol, or
(r) a grassy odor, such as by preference 2-ethyl pyridine, 2,6-dimethyl naphthalene, hexanal, and/or (z)-3-hexenol, or
(s) a green note, by preference 2-ethylhexanol, 6-decenal, dimethyl heptenal, hexanol, heptanol, methyl 2-butenal, hexyl octanoate, nonanoic acid, undecanone, methyl geranate, isobornyl formate, butanal, octanal, nonanal, epoxy 2-decenal, cis-linalool, pyranoxide, nonanol, alpha, γ-dimethyl allyl alcohol, (Z)-2-penten-1-ol, (Z)-3-hexenyl butanoate, isobutyl thiazol, (E)-2-nonenal, 2-dodecenal, (Z)-4-decenal, 2-octenal, 2-hepten-1-al, bicyclogermacrene, α-thujene, (Z)-β-farnesene, (−)-γ-elemene, 2,4-octadienal, fucoserratene, hexenyl acetate, geranyl acetone, valencene, β-eudesmol, 1-hexenol, (E)-2-undecenal, artemisia ketone, viridiflorol, 2,6-nonadienal, trimethylphenylbutenone, 2,4-nonadienal, butyl isothiocyanate, 2-pentanol, elemol, 2-hexenal, 3-hexenal, (+)-(E)-limonene oxide, cis-isocitral, dimethyl octadienal, bornyl formate, bornyl isovalerate, isobutyraldehyde, 2,4-hexadienal, nonanone, (E)-2-hexenal, (+)-cis-rose oxide, menthone, coumarin, (epoxymethylbutyl) methyl furan, 2-hexenol, (E)-2-hexenol, and/or carvyl acetate, or
(t) a green-tea-like odor, by preference (−)-cubenol, or
(u) an herbal odor, by preference octanone, hexyl octanoate, caryophyllene oxide, methyl butenol, safranal, benzyl benzoate, bornyl butyrate, hexyl acetate, β-bisabolol, piperitol, β-selinene, α-cubebene, p-menth-1-en-9-ol, 1,5,9,9-tetramethyl-12-oxabicyclododeca-4,7-diene, T-muurolol, (−)-cubenol, levomenol, ocimene, α-thujene, p-menth-1-en-9-yl acetate, dehydrocarveol, artemisia alcohol, γ-muurolene, hydroxypentanone, (Z)-ocimene, β-elemene, δ-cadinol, (E)-β-ocimene, (Z)-dihydrocarvone, α-cadinol, calamenene, (Z)-piperitol, lavandulol, β-bourbonene, (Z)-3-hexenyl 2-methyl butanoate, 4-(1-methyl ethyl) benzene methanol, artemisia ketone, methyl 2-butenol, heptanol, (E)-dihydrocarvone, p-2-menthen-1-ol, α-curcumene, spathulenol, sesquiphellandrene, citronellyl valerate, bornyl isovalerate, 1,5-octadien-3-ol, methyl benzoate, 2,3,4,5-tetrahydroanisole, and/or hydroxycalamenene, or
(v) a honey-like odor, by preference ethyl cinnamate, β-phenethyl acetate, phenylacetic acid, phenyl ethanal, methyl anthranilate, cinnamic acid, β-damascenone, ethyl-(E)-cinnamate, 2-phenyl ethyl alcohol, citronellyl valerate, phenyl ethyl benzoate, and/or eugenol, or
(w) a hyacinth-like odor, by preference hotrienol, or
(x) a jasmine-like odor, by preference methyl jasmonate, methyl dihydroepijasmonate, and/or methyl epijasmonate, or
(y) a lavender-like odor, by preference linalyl valerate and/or linalool, or
(z) a lemony odor, by preference neral, octanal, δ-3-carene, limonene, geranial, 4-mercapto-4-methyl-2-pentanol, citral, 2,3-dehydro-1,8-cineol, and/or α-terpinene, or
(aa) a lily-like odor, by preference dodecanal, or
(bb) a magnolia-like odor, by preference geranyl acetone, or
(cc) a tangerine-like odor, by preference undecanol, or
(dd) a melon-like odor, by preference dimethyl heptenal, or
(ee) a minty odor, by preference menthone, ethyl salicylate, p-anisaldehyde, 2,4,5,7a-tetrahydro-3,6-dimethylbenzofuran, epoxy-p-menthene, geranial, (methylbutenyl)methylfuran, dihydrocarvyl acetate, β-cyclocitral, 1,8-cineol, β-phellandrene, methyl pentanone, (+)-limonene, dihydrocarveol, (−)-carvone, (E)-p-mentha-2,8-dien-1-ol, isopulegyl acetate, piperitone, 2,3-dehydro-1,8-cineol, α-terpineol, DL-carvone, and/or α-phellandrene, or
(ff) a nutty odor, by preference 5-methyl-(E)-2-hepten-4-on, γ-heptalactone, 2-acetylpyrrole, 3-octen-2-one, dihydromethyl cyclopentapyrazine, acetylthiazol, 2-octenal, 2,4-heptadienal, 3-octenone, hydroxypentanone, octanol, dimethylpyrazine, methylquinoxaline, and/or acetylpyrroline, or
(gg) an orange-like odor, by preference methyl octanoate, undecanone, decyl alcohol, limonene, and/or 2-decenal, or
(hh) an orange-peel-like odor, by preference decanal and/or β-carene, or
(ii) a peach-like odor, by preference γ-nonalactone, (Z)-6-dodecene-γ-lactone, δ-decalacton, R-δ-decenolactone, hexyl hexanoate, 5-octanolide, γ-decalactone, and/or α-undecalactone, or
(jj) a peppermint-like odor, by preference methyl salicylate and/or l-menthol, or
(kk) a pine-like odor, by preference α-p-dimethylstyrene, β-pinene, bornyl benzoate, δ-terpinene, dihydroterpinyl acetate, and/or α-pinene, or
(ll) a pineapple-like odor, by preference propyl butyrate, propyl propanoate, and/or ethyl acetate, or
(mm) a plum-like odor, by preference benzyl butanoate, or
(nn) a raspberry-like odor, by preference β-ionone, or
(oo) a rose-like odor, by preference β-phenethyl acetate, 2-ethylhexanol, geranyl valerate, geranyl acetate, citronellol, geraniol, geranyl butyrate, geranyl isovalerate, citronellyl butyrate, citronellyl acetate, isogeraniol, tetrahydro-4-methyl-2-(2-methyl-1-propenyl)-2,5-cis-2H-pyran, isogeraniol, 2-phenyl ethyl alcohol, citronellyl valerate, and/or citronellyl isobutyrate, or
(pp) a green-mint-like odor, by preference carvyl acetate and/or carveol, or
(qq) a strawberry-like odor, by preference hexylmethyl butyrate, methyl cinnamate, pentenal, or
(rr) a sweetish odor, by preference benzyl alcohol, ethyl phenyl acetate, tridecanal, nerol, methyl hexanoate, linalyl isovalerate, undecanaldehyde, caryophyllene oxide, linalyl acetate, safranal, uncineol, phenyl ethanal, p-anisaldehyde, eudesmol, ethylmethylpyrazine, citronellyl butyrate, 4-methyl-3-penten-2-one, nonyl acetate, 10-epi-γ-eudesmol, β-bisabolol, (z)-6-dodecene-γ-lactone, β-farnesene, 2-dodecenal, γ-dodecalactone, epoxy-β-ionone, 2-undecenal, styrene glycol, methyl furaneol, (−)-cis-rose oxide, (E)-β-ocimene, dimethylmethoxyfuranone, 1,8-cineole, ethyl benzaldehyde, 2-pentylthiophene, α-farnesene, methionol, 7-methoxycoumarin, (Z)-3-hexenyl-2-methyl butanoate, o-aminoacetophenone, viridiflorol, isopiperitone, β-sinensal, ethyl vanillate, methyl butanoate, p-methoxystyrene, 6-methoxyeugenol, 4-hexanolide, 6-dodecalactone, sesquiphellandrene, diethyl malate, linalyl butyrate, guaiacol, coumarin, methyl benzoate, isopropyl benzoate, safrole, durene, γ-butyrolactone, ethyl isobutyrate, and/or furfural, or
(ss) a vanilla-like odor, by preference vanillin, methyl vanillate, acetovanillone, and/or ethyl vanillate, or
(tt) a watermelon-like odor, by preference 2,4-nonadienal, or
(uu) a woody odor, by preference α-muurolene, cadina-1,4-dien-3-ol, isocaryophyllene, eudesmol, α-ionone, bornyl butyrate, (E)-α-bergamotene, linalool oxide, ethylpyrazine, 10-epi-γ-eudesmol, germacrene B, trans-sabinene hydrate, dihydrolinalool, isodihydrocarveol, β-farnesene, β-sesquiphellandrene, δ-elemene, α-calacorene, epoxy-β-ionone, germacrene D, bicyclogermacrene, alloaromadendrene, α-thujene, oxo-β-ionone, (−)-γ-elemene, γ-muurolene, sabinene, α-guaiene, α-copaene, γ-cadinene, nerolidol, β-eudesmol, α-cadinol, δ-cadinene, 4,5-dimethoxy-6-(2-propenyl)-1,3-benzodioxol, [1ar-(1aalpha,4aalpha,7alpha,7abeta,7balpha)]-decahydro-1,1,7-trimethyl-4-methylene-1H-cycloprop[e]azulene, α-gurjunene, guaiol, α-farnesene, γ-selinene, 4-(1-methylethyl)benzenemethanol, perillene, elemol, α-humulene, β-caryophyllene, and/or β-guaiene, or
(vv) mixtures of the aforesaid.
   The aforesaid odorants can by preference be contained in the particles according to the present invention. They are suitable to a high degree in particular for the scenting of washing, cleaning, or care agents. Perfume stability can also be even further improved, according to the present invention, in connection with the aforesaid odorants.

As has become clear from the description above, zeolite in particular can stand in the way of perfume stability. For this reason, according to a preferred embodiment the particles according to the present invention contain less than 25 wt % zeolite, based on the entire particle. By preference, zeolite is contained in fact only in a quantity of less than 20 wt %, advantageously less than 15 wt %, in more greatly advantageous fashion less than 10 wt %, in more advantageous fashion less than 5 wt %, based on the entire particle.

Advantageously, the upper zeolite limit can also lie between the aforesaid values, i.e. for example at a value of, by preference, 24 wt %, 23 wt %, 22 wt %, 21 wt %, 19 wt %, 18 wt %, 17 wt %, 16 wt %, 14 wt %, 13 wt %, 12 wt %, 11 wt %, 9 wt %, 8 wt %, 7 wt %, 6 wt %, 4 wt %, 3 wt %, 2 wt %, or 1 wt %, based on the entire particle.

In a preferred embodiment, however, the particle can also contain specific minimum values of zeolite, namely at least 1 wt %, advantageously at least 5 wt %, in more greatly advantageous fashion at least 10 wt %, by preference at least 15 wt %, in particular at least 20 wt % zeolite, based on the entire particle.

It may be advantageous in overall terms if the zeolite quantity lies between the aforementioned minimum and maximum quantities, i.e. for example in a range from 1 to 25 wt % or 5 to 20 wt % zeolite, or 1 to 15 wt %, or in another range according to another possible combination of the values just recited.

According to a further preferred embodiment, the zeolite is by preference zeolite X, Y, A, P, MAP, and/or mixtures thereof. The zeolite should contain by preference less than 25 wt %, advantageously less than 20 wt %, in more greatly advantageous fashion less than 15 wt %, in even more advantageous fashion less than 8 wt %, in particular less than 5 wt % desorbable water. A zeolite of this kind can be obtained, for example, by activating or dehydrating the zeolite at temperatures from 150° C. to 350° C., if applicable at reduced pressure (advantageously from approx. 0.001 to approx. 20 torr). The term then used is, for example, activated/dehydrated zeolite.

According to a preferred embodiment, the particle according to the present invention is entirely zeolite-free, i.e. contains 0 wt % zeolite.

In a preferred embodiment, the particle contains specific minimum values of perfume, namely at least 1 wt %, advantageously at least 2 wt %, in appreciably advantageous fashion at least 3 wt %, in more advantageous fashion at least 4 wt %, in more greatly advantageous fashion at least 5 wt %, in even more greatly advantageous fashion at least 6 wt %, in very advantageous fashion at least 7 wt %, in particularly advantageous fashion at least 8 wt %, in very particularly advantageous fashion at least 10 wt %, in considerably advantageous fashion at least 11 wt %, in very considerably advantageous fashion at least 12 wt %, in extremely advantageous fashion at least 13 wt %, in most highly advantageous fashion at least 14 wt %, in exceedingly advantageous fashion at least 16 wt %, in exceptionally advantageous fashion at least 18 wt %, in unusually advantageous fashion at least 20 wt %, in extraordinarily advantageous fashion at least 22 wt %, in particular at least 24 wt % perfume, based on the entire particle.

Advantageously, the particles according to the present invention permit very large quantities of perfume to be accepted without negatively affecting good particle properties such as, for example, pourability. Advantageously, clumping does not occur. Surprisingly, perfume stability is ensured in very good fashion in the particle according to the present invention even with high perfume loading, i.e. for example with perfume quantities of at least 5 wt %, at least 10 wt %, at least 15 wt %, at least 20 wt %, etc., based on the entire particle.

In a preferred embodiment, the particle contains nonionic surfactant, by preference selected from the group of the alkoxylated alcohols, the alkyl phenol polyglycol ethers, the alkoxylated fatty acid alkyl esters, the polyhydroxy fatty acid amides, the alkyl glycosides, the alkylpolyglucosides, the amine oxides, and/or the long-chain alkyl sulfoxides, in particular in a quantity of at least 0.1 wt % based on the entire particle.

The particle is by preference impregnated with the nonionic surfactant, i.e. the nonionic surfactant is advantageously substantially distributed in the carrier material.

When the particles contain nonionic surfactant, this then advantageously results in an even more intensive scent experience for the consumer, for example when washing laundry with a detergent formulation that contains the particles according to the present invention. The consumer can therefore, advantageously perceive a more intensive scent of the washed laundry as compared with laundry that was washed with a conventionally perfumed detergent formulation, even if the absolute quantity of perfume contained was the same. A scent-intensifying effect is therefore achieved here which directly affects the particles as well as objects into which said particles are incorporated, for example detergent formulations, as well as things such as, for example, textiles that are treated with the objects (in this case, a detergent formulation).

A further advantage of the particles according to the present invention that additionally contain nonionic surfactant is, surprisingly, the fact that the perfume component incorporated into the particle is even further stabilized. Perfume decomposition tendencies are therefore even further suppressed. The perfume-stabilizing effect according to the present invention is thus further intensified. This is also true, in particular, when the particle is incorporated into an object such as, for example, into a detergent formulation, that, because of its object property (e.g. its alkalinity) is fairly detrimental to the stability of perfume. Here as well, the additional perfume-stabilizing effect has an even further improving action.

A further advantage of the particles according to the invention that additionally contain nonionic surfactant is, surprisingly, the fact that the scent impression resulting from the particles lasts even longer both indirectly and directly. “Directly” means in this connection that the particles as such according to the present invention are fragrant over a longer period of time. “Indirectly” means in this connection that objects (e.g. a detergent formulation) that contain the particles according to the present invention are fragrant for longer, and that in fact when said objects (e.g. a detergent formulation for washing textiles) are used, the things (in this case, a washed textile) treated therewith are fragrant for longer.

The scent retardation according to the present invention is thus further intensified, this scent-retarding action (i.e. the extension over time of the scent impression) referring both to the particles and to objects containing the particles, and to the things treated with said objects.

A further advantage of the particles according to the present invention that additionally contain nonionic surfactant is also, surprisingly, that the addition or presence of nonionic surfactant makes it possible to load the carrier material of the particles with even greater perfume quantities. This is surprising most of all because it might have been assumed that the perfume quantity receivable by the carrier material would have to decrease if the carrier material additionally needs to accept a certain quantity of a further substance. A further improvement, in particular in fact a maximization, of the perfume acceptance capability of the carrier material is, however, achieved, so that perfume loading of the carrier material to an even greater extent becomes possible.

The usable quantity of nonionic surfactant can be advantageously adjusted: In a preferred embodiment, the particle according to the present invention contains advantageously at least 0.2 wt %, in more advantageous fashion at least 0.3 wt %, in even more advantageous fashion at least 0.4 wt %, in more greatly advantageous fashion at least 0.5 wt %, in even more greatly advantageous fashion at least 0.6 wt %, in very advantageous fashion at least 0.7 wt %, in particularly advantageous fashion at least 0.8 wt %, in very particularly advantageous fashion at least 0.9 wt %, in considerably advantageous fashion at least 1.0 wt %, in very considerably advantageous fashion at least 1.1 wt %, in extremely advantageous fashion at least 1.2 wt %, in most highly advantageous fashion at least 1.3 wt %, exceedingly advantageously at least 1.4 wt %, in exceptionally advantageous fashion at least 1.5 wt %, in unusually advantageous fashion at least 1.6 wt %, in extraordinarily advantageous fashion at least 1.7 wt %, in particular at least 1.8 wt % nonionic surfactant, based on the entire particle.

According to another preferred embodiment, however, it may also be advantageous that the particle does not exceed certain maximum quantities of nonionic surfactant, i.e. less than 30 wt %, advantageously less than 26 wt %, in appreciable advantageous fashion less than 24 wt %, in more advantageous fashion less than 22 wt %, in more greatly advantageous fashion less than 20 wt %, in even more greatly advantageous fashion less than 18 wt %, in very advantageous fashion less than 16 wt %, in particularly advantageous fashion less than 14 wt %, in very particularly advantageous fashion less than 12 wt %, in considerably advantageous fashion less than 11 wt %, in very considerably advantageous fashion less than 10 wt %, in extremely advantageous fashion less than 9 wt %, in very highly advantageous fashion less than 8 wt %, in exceedingly advantageous fashion less than 7 wt %, in exceptionally advantageous fashion less than 6 wt %, in unusually advantageous fashion less than 5 wt %, in extraordinarily advantageous fashion less than 4 wt %, in particular less than 3 wt % nonionic surfactant, based on the entire particle.

According to a further preferred embodiment, alkoxylated alcohol is contain at least in part as a nonionic surfactant, by preference in quantities of at least 40 wt %, advantageously at least 50 wt %, in more greatly advantageous fashion at least 60 wt %, in exceedingly advantageous fashion at least 70 wt %, in even more advantageous fashion at least 80 wt %, in particular at least 90 wt %, in most advantageous fashion in quantities of 100 wt %, based in each case on the total quantity of nonionic surfactant that is contained in the particle, the alcohols advantageously being ethoxylated, in particular primary alcohols having by preference 8 to 20, in particular 12 to 18 carbon atoms and by preference an average of 1 to 12 mol alkylene oxide, by preference ethylene oxide, per mol of alcohol.

According to a further preferred embodiment, the nonionic surfactants are a mixture of at least two different nonionic surfactants, by preference of at least two different alkoxylated, advantageously ethoxylated, in particular primary alcohols, the distinguishing feature in terms of the alkoxylated alcohols being by preference the degree of alkoxylation.

If at least one alkoxylated, by preference ethoxylated alcohol having a degree of alkoxylation less than 7, advantageously no greater than 6, more greatly advantageously no greater than 5, in particular no greater than 4.5, and at least one further alkoxylated, advantageously ethoxylated alcohol having a degree of alkoxylation of at least 7, are present in this mixture of at least two different nonionic surfactants, this is then a further preferred embodiment of the invention.

According to a further preferred embodiment of the invention, the ratio of lower-alkoxylated alcohol to higher-alkoxylated alcohol is in the range from 5:1 to 1:5, by preference from 4:1 to 1:4, advantageously 3:1 to 1:3, in particular 2:1 to 1:2.

An essential constituent of the particle according to the present invention, in addition to the layered silicate and the perfume, is the carbonate and the sulfate.

In the context of the carbonates, the water-soluble ones are particularly preferred. Advantageously,
f) alkali carbonates such as, by preference, sodium carbonate and/or potassium carbonate;
g) alkaline earth carbonates such as, by preference, magnesium carbonate;
h) hydrogencarbonates such as, by preference, sodium hydrogencarbonate, potassium hydrogencarbonate, and/or ammonium hydrogencarbonate;
i) sesquicarbonates such as, by preference, sodium sesquicarbonate (Na₂CO₃ NaHCO₃.2H₂O) and/or potassium sesquicarbonate (K₂CO₃.KHCO₃.2H₂O);
j) other carbonates such as, for example, ammonium carbonate;
k) mixtures of the aforesaid,
   can be used.

It may be advantageous to use the carbonate within a certain bandwidth of upper and/or lower limits.

In a preferred embodiment, the particle contains certain minimum values of carbonate, namely at least 5 wt %, advantageously at least 10 wt %, in appreciably advantageous fashion at least 12 wt %, in more advantageous fashion at least 14 wt %, in more greatly advantageous fashion at least 16 wt %, in even more greatly advantageous fashion at least 18 wt %, in very advantageous fashion at least 20 wt %, in particular 22 wt %, in considerably advantageous fashion at least 24 wt %, in very considerably advantageous fashion 26 wt %, in extremely advantageous fashion 28 wt %, in very highly advantageous fashion at least 30 wt %, in exceedingly advantageous fashion at least 32 wt %, in exceptionally advantageous fashion at least 34 wt %, in unusually advantageous fashion at least 36 wt %, in extraordinarily advantageous fashion at least 38 wt %, in particular 40 wt % carbonate, based on the entire particle.

High carbonate values are also advantageous because they bring about, for the washing agent into which the particles can be incorporated, an improved alkalinity as well as an improvement in washing action due to electrolyte effects.

The maximum quantity of carbonate can also advantageously be established. According to another preferred embodiment, it may therefore be advantageous for the particles not to exceed certain maximum quantities of carbonate, i.e. less than 70 wt %, advantageously less than 65 wt %, in more greatly advantageous fashion less than 60 wt %, in very advantageous fashion less than 58 wt %, in particularly advantageous fashion less than 56 wt %, in very particularly advantageous fashion less than 54 wt %, in considerably advantageous fashion less than 52 wt %, in very considerably advantageous fashion less than 50 wt %, in extremely advantageous fashion less than 48 wt %, in very highly advantageous fashion less than 46 wt %, in exceedingly advantageous fashion less than 44 wt %, in particular less than 42 wt % carbonate, based on the entire particle.

It is advantageous in global terms if the carbonate quantity lies within the aforementioned minimum and maximum values, i.e. for example in a range from 5 to 70 wt % or 10 to 60 wt % or 25 to 50 wt %, or in a range according to another possible combination of the values just recited.

In the context of the sulfates, the water-soluble ones are particularly preferred. Advantageously,
l) alkali sulfates such as, by preference, sodium sulfate and/or potassium sulfate;
m) alkaline earth sulfates such as, by preference, magnesium sulfate;
n) hydrogensulfates such as, by preference, sodium hydrogensulfate and/or potassium hydrogensulfate, ammonium hydrogensulfate;
o) other sulfates such as, for example, ammonium sulfate;
p) mixtures of the aforesaid,
   can be used.

It may be advantageous to use the sulfate within a certain bandwidth of upper and/or lower limits.

In a preferred embodiment, the particle contains certain minimum values of sulfate, namely at least 5 wt %, advantageously at least 10 wt %, in substantially advantageous fashion at least 12 wt %, in more advantageous fashion at least 14 wt %, in more greatly advantageous fashion at least 16 wt %, in even more greatly advantageous fashion at least 18 wt %, in very advantageous fashion at least 20 wt %, in particular 22 wt %, in considerably advantageous fashion at least 24 wt %, in very considerably advantageous fashion 26 wt %, in extremely advantageous fashion 28 wt %, in very highly advantageous fashion at least 30 wt %, in exceedingly advantageous fashion at least 32 wt %, in exceptionally advantageous fashion at least 34 wt %, in unusually advantageous fashion at least 36 wt %, in extraordinarily advantageous fashion at least 38 wt %, in particular 40 wt % sulfate, based on the entire particle. High sulfate contents are also advantageous because they impart to the particles even better pourability, even better dispensing qualities, and even better solubility.

The maximum quantity of sulfate can also advantageously be established. According to another preferred embodiment, it may therefore be advantageous for the particles not to exceed certain maximum quantities of sulfate, i.e. no more than 70 wt %, advantageously less than 68 wt %, in more greatly advantageous fashion less than 66 wt %, in very advantageous fashion less than 64 wt %, in particularly advantageous fashion less than 62 wt %, in very particularly advantageous fashion less than 60 wt %, in considerably advantageous fashion less than 58 wt %, in very considerably advantageous fashion less than 56 wt %, in extremely advantageous fashion less than 54 wt %, in very highly advantageous fashion less than 52 wt %, in exceedingly advantageous fashion no more than 50 wt %, in particular less than 48 wt % sulfate, based on the entire particle.

It is advantageous in global terms if the sulfate quantity lies within the aforementioned minimum and maximum values, i.e. for example in a range from 5 to 70 wt % or 10 to 60 wt % or 25 to 50 wt %, or in a range according to another possible combination of the aforesaid values from 5 to 70 wt %.

According to the present invention, the ratio of layered silicate to the total quantity of sulfate and carbonate in the particle according to the present invention is ≦1:2. According to a preferred embodiment, the ratio of layered silicate to the total quantity of sulfate and carbonate is ≦2:5, by preference ≦1:3, advantageously ≦2:7, in more advantageous fashion ≦1:4, in particular ≦1:5.

It may also be advantageous to establish a certain ratio of carbonate to sulfate. According to a preferred embodiment, the ratio of carbonate to sulfate is in the range from 5:1 to 1:1, by preference in the range from 4:1 to 1:1, advantageously in the range from 3:1 to 1:1, in particular in the range from 2:1 to 1:1. According to another, likewise preferred embodiment, the ratio of sulfate to carbonate is in the range from 5:1 to 1:1, by preference in the range from 4:1 to 1:1, advantageously in the range from 3:1 to 1:1, in particular in the range from 2:1 to 1:1.

It may also be advantageous to establish a certain ratio of layered silicate to carbonate. Advantageously, according to a preferred embodiment a ratio of layered silicate to carbonate of ≦1:2 exists in the particle according to the present invention. According to a preferred embodiment, the ratio of layered silicate to carbonate is ≦2:5, by preference ≦1:3, advantageously ≦2:7, in more advantageous fashion ≦1:4, in particular ≦1:5.

According to a further preferred embodiment, the particle according to the present invention contains carbonate(s) and sulfate(s) in a total quantity of >10 wt %, >15 wt %, >20 wt %, >25 wt %, >30 wt %, >35 wt %, >40 wt %, >45 wt %, >50 wt %, >55 wt %, >60 wt %, >65 wt %, or >70 wt %, based on the entire particle.

It may also be advantageous to establish a certain ratio of layered silicate to sulfate. Advantageously, according to a preferred embodiment a ratio of layered silicate to sulfate of ≦1:2 exists in the particle according to the present invention. According to a preferred embodiment, the ratio of layered silicate to sulfate is ≦2:5, by preference ≦1:3, advantageously ≦2:7, in more advantageous fashion ≦1:4, in particular ≦1:5.

According to a further preferred embodiment, the particle according to the present invention contains a polymeric clay flocculent, the clay flocculent being a polymer or copolymer that by preference is derived from monomers that are selected from among ethylene oxide, acrylamide, acrylic acid, dimethyl aminoethyl methacrylate, vinyl alcohol, vinylpyrrolidone, ethyleneimine, and mixtures thereof, possessing in particular a weight-averaged molecular weight from 100,000 to 10 million, and being contained by preference in the range from 0.005 wt % to 20 wt %, based on the layered silicate in the particle.

Advantageously, the textile-softening layered silicates are thus deposited even more efficiently on the textile during the washing process. Deposition is enhanced, and proceeds even more uniformly.

According to a preferred embodiment, the polymeric clay flocculent is obtained from monomers that are selected from among ethylene oxide, acrylamide, and acrylic acid.

According to a preferred embodiment, the polymeric clay flocculant has a weight-averaged molecular weight from 150,000 to 5 million, by preference from 150,000 to 800,000.

According to a preferred embodiment, the polymeric clay flocculent has a molecular weight from 150,000 to 800,000. According to another preferred embodiment, the clay flocculent possesses a molecular weight from 800,000 to 5 million.

According to a preferred embodiment, the polymeric clay flocculent is present in a quantity from 0.005 wt % to 10 wt %, by preference from 0.005 wt % to 5 wt %, in particular from 0.005 wt % to 2 wt %, based on the layered silicate.

By preference, the particle according to the present invention can also contain further ingredients, and according to a preferred embodiment the particle contains cationic surfactant, zwitterionic compounds, ampholytes, amphosurfactants, betaines, cationic polymers, and/or amphoteric polymers. When such substances are present, more greatly improved object scenting can be achieved with use of the particles, including in particular a more greatly improved scent retardation. This means that, for example, when textile objects are washed with a washing agent that contains such particles according to the present invention, it is then possible for even more scent to remain adhering to the textile objects, and for it also to last longer there.

According to a preferred embodiment, the particle contains at least one quaternary ammonium compound, by preference an alkylated quaternary ammonium compound, in particular in quantities from 0.1 wt % to 30 wt % based on the entire particle.

According to a preferred embodiment, the particle contains a quaternary ammonium compound of formula (I)

R¹(R²)(R³)(R⁴)N⁺X⁻, (I)

where R¹, R², and R³, mutually independently, are selected from C₁-C₄ alkyl, C₁-C₄ hydroxyalkyl, benzyl, and —(C₂H₄O)ₓH, with x equal to 2 to 5, and wherein R⁴ is a C₈-C₂₂ alkyl, and where X⁻ is an anion, by preference a halide, methosulfate, methophosphate, or phosphate ion, as well as mixtures thereof.

According to a preferred embodiment, the particle contains a quaternary ammonium compound of formula (II)

R⁵R⁶ₙR⁷₃₋ₙN+X⁻, (II)

where R⁵ is a C₆-C₂₄ alkyl or alkenyl, where each R⁶, mutually independently, is a —(CₙH₂O)ₓR⁸ group, with n equal to 1 to 4 and with x equal to 1 to 14, and where R⁸ is a methyl, ethyl, or preferably a hydrogen, and where each R⁷, mutually independently, is a C₁-C₂ alkyl or alkenyl group, with m equal to 1 to 3, and where X⁻ is an anion, by preference a halide, methosulfate, methophosphate, or phosphate ion, and mixtures thereof. According to a preferred embodiment, R⁶ is a —CH₂CH₂OH, R⁷, mutually independently, is a C₁-C₄ alkyl, with m equal to 1 or 2, and where R⁵ is a linear C₆-C₁₄ alkyl group.

According to a preferred embodiment, the particle contains a C₈-C₁₆ alkyl(dihydroxyethyl)methylammonium compound, by preference a C₁₂-C₁₄ alkyl(dihydroxyethyl)methylammonium compound, and/or a C₈-C₁₆ alkyl(hydroxyethyl)dimethylammonium compound, by preference a C₁₂-C₁₄ alkyl(hydroxyethyl)dimethylammonium compound, in particular the respective halides, methosulfates, methophosphates, or phosphates, as well as mixtures thereof.

Zwitterionic compounds can also be used with advantage. According to a preferred embodiment, the particle contains a zwitterionic compound of formula (III):

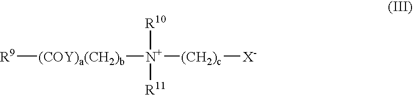

where the R⁹ denotes a C₆₋₂₈ alkyl or alkenyl group, and where R¹⁰ and R¹¹ each, mutually independently, are C₁₋₄ alkyl groups, and where a denotes the number 0 or 1, b and c are each selected, mutually independently, from whole numbers from 1 to 4, and where Y is oxygen or nitrogen, and where X is an atom or atom group having a negative charge. The negative charge is usually localized on oxygen atoms by emission of a proton from carboxy or sulfo groups, phosphoric acid radicals, acid phenolic or enolic hydroxy groups.

According to a preferred embodiment, the particle contains at least one alkylamidoalkylene dimethylcarboxylic acid betaine of formula (IV):

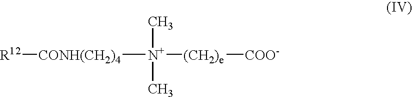

where d and e, mutually independently, are whole numbers from 1 to 4, by preference d is equal to 2 or 3, and e equal to 2 or 3, and where R¹² denotes a C₁₀₋₁₈ alkyl chain, or mixtures thereof.

Cationic nitrile can also be used with advantage. According to a preferred embodiment, the particle contains at least one cationic nitrile of formula (V)

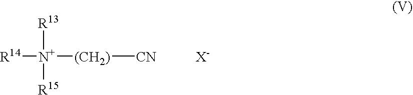

in which R¹³ denotes —H, —CH₃, a C₂₋₂₄ alkyl or alkenyl radical, a substituted C₂₋₂₄ alkyl or alkenyl radical having at least one substituent from the group —Cl, —Br, —OH, —NH₂, —CN, an alkyl or alkenylaryl radical having a C₁₋₂₄ alkyl group, or a substituted alkyl or alkenylaryl radical having a C₁₋₂₄ alkyl group and at least one further substituent on the aromatic ring, R¹⁴ and R¹⁵ are selected, mutually independently, from —CH₂—CN, —CH₃, —CH₂—CH₃, —CH₂—CH₂—CH₃, —CH(CH₃)—CH₃, —CH₂—OH, —CH₂—CH₂—OH, —CH(OH)—CH₃, —CH₂—CH₂—CH₂—OH, —CH₂—CH(OH)—CH₃, —CH(OH)—CH₂—CH₃, —(CH₂CH₂—O)ₙH where n=1, 2, 3, 4, 5 or 6, and X is an anion. According to a preferred embodiment, X⁻ denotes an anion that is selected from the group of chloride, bromide, iodide, hydrogensulfate, methosulfate, lauryl sulfate, dodecylbenzenesulfonate, p-toluenesulfonate (tosylate), cumenesulfonate, or xylenesulfonate, or mixtures thereof.

According to a preferred embodiment, the particle contains at least one cationic nitrile of formula (VI)

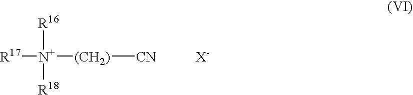

in which R¹⁶, R¹⁷, and R¹⁸ are selected, mutually independently, from —CH₃, —CH₂—CH₃, —CH₂—CH₂—CH₃, —CH(CH₃)—CH₃, where R⁶ can additionally also be —H and X is an anion, where by preference R⁷=R⁸=—CH₃ and in particular R¹⁶=R¹⁷=R¹⁸=—CH₃, and compounds of the formulas (CH₃)₃N⁽⁺⁾CH₂—CNX⁻, (CH₃CH₂)₃N⁽⁺⁾CH₂—CNX⁻, (CH₃CH₂CH₂)₃N(⁺)CH₂—CNX⁻, (CH₃CH(CH₃))₃N⁽⁺⁾CH₂—CNX⁻, or (HO—CH₂—CH₂)₃N⁽⁺⁾CH₂—CNX⁻ are particularly preferred. According to a preferred embodiment, X⁻ denotes an anion selected from the group of chloride, bromide, iodide, sulfate, hydrogensulfate, methosulfate, lauryl sulfate, dodecylbenzenesulfonate, p-toluenesulfonate (tosylate), cumenesulfonate, or xylenesulfonate, or mixtures thereof.

Amides can also be used with advantage. According to a preferred embodiment, the particle contains an amide, by preference an amide of the formula R₁₉R₂₀NCOR₂₁, in which R₁₉ and R₂₀ are selected, mutually independently, from among C₁-C₂₂ alkyl, C₁-C₂₂ alkenyl, C₁-C₂₂ hydroxyalkyl, aryl, and alkylaryl groups; R₂, denotes hydrogen or a C₁-C₂₂ alkyl, C₁-C₂₂ alkenyl, aryl, or alkylaryl group, or is O—R₂₂, in which R₂₂ signifies a C₁-C₂₂ alkyl, C₁-C₂₂ alkenyl, an aryl or alkylaryl group, the amide being contained in particular in quantities from 1% to 10% based on the entire particle

Imidazoline (derivatives) can also be used with advantage. According to a preferred embodiment, the particle contains imidazoline (derivatives), by preference imidazolines of the formula 1-(R₂₃)amido(R₂₄)-2-(R₂₅)-imidazoline, in which R₂₃, R₂₅ are selected, mutually independently, from C₁₂-C₂₂ alkyl and R₂₄ is selected from C₁-C₄ alkyl, in particular in quantities from 1 wt % to 10 wt % based on the entire particle.

Organic humectants can also be used with advantage, since they result in more efficient deposition of the textile-softening layered silicates onto the textiles. According to a preferred embodiment, the particle contains organic humectant that is selected in particular from glycerol, ethylene glycol, propylene glycol, dimers and trimers of glycerol, and/or mixtures thereof, by preference in quantities from 0.1 wt % to 30 wt % based on the entire particle.

Complexing agents can also be used with advantage. According to a preferred embodiment, the particle contains a complexing agent, by preference phosphonate and/or a citrate, in particular in quantities from 0.1 wt % to 10 wt % based on the entire particle.

Pentaerythrite (derivatives) can also be used with advantage, since they further improve the laundry softening effect, e.g. that of the layered silicates. According to a preferred embodiment, the particle contains pentaerythrite (derivatives), such as by preference a C₂-C₂₄ aliphatic acid ester of pentaerythrite, in particular in quantities from 0.1 to 30 wt % based on the entire particle.

Alkali silicate can also be used with advantage. According to a preferred embodiment, the particle contains an alkali silicate, by preference having an M₂O:SiO₂ modulus in the range from 1:1.9 to 1:3.3, M denoting an alkali-metal ion. According to a preferred embodiment, the particle contains amorphous sodium silicate by preference having a Na₂O:SiO₂ modulus in the range from 1:2 to 1:2.8.

The particles can additionally contain further carrier material, selected by preference from
(a) silicic acids, by preference precipitated silicic acids, in particular the silica gels, which advantageously are hydrophobic or hydrophilic, and/or
(b) carrier materials from the group of the surfactants, surfactant compounds, citrates, alkali metal phosphates, chitin microspheres, pectin, gums, gelatins, resins, starches, in particular porous starches, modified starches, and/or carboxylalkyl starches, di- and/or polysaccharides, cyclodextrins, maltodextrins, (co)polymers, by preference synthetic (co)polymers, in particular water-soluble (co)polymers and/or terpolymers, and/or mixtures thereof.

The carrier material can accordingly also comprise, at least in part, one or more (co)polymers as a carrier material, which by preference are selected at least in part from the following groups:
a) homopolymers, selected from polyvinyl compounds such as, by preference, polyvinyl acetates, polyvinyl alcohol, and/or polyvinylpyrrolidone, polycarboxylic acids such as, by preference, polyacrylic acid and/or polymethacrylic acid; polysulfonic acids such as, by preference, polystyrenesulfonic acids; polyesters, such as by preference glycol polyacrylates; polyamides; polyacrylamides; polyurethanes, by preference polyurethanes that carry ionic groups, for example carboxy groups, sulfonic acid groups, or tertiary amines, or polyurethanes that by preference contain nonionic hydrophilic groups, such as ethylene oxide, polyethylene oxide, polypropylene oxide, and polyalkylene glycol derivatives;
b) polycondensates, such as by preference ethyoxylated phenol, formaldehyde resins, preferably sulfonated aromatic formaldehyde resins, urea, or melamines, formaldehyde compounds, polyamides, polyamines, and epichlorohydrin resins;
c) AB copolymers, in which A represents a more-water-soluble group or one swellable in water, and B a less-water-soluble group or one less swellable in water, by preference selected from styrene copolymers, such as in particular styrene-acrylic acid polymers or styrene-ethylene oxide polymers, copolymers of polyvinyl and maleic acid compounds, such as by preference styrene-maleic acid anhydride polymers or vinyl acetates, maleic acid ester polymers, polyvinyl-polyalkylene copolymers, such as by preference vinyl acetate-ethylene polymers, ethylene-acrylic acid-acrylic acid ester polymers or ethylene-acrylic acid-acrylonitrile polymers, vinyl copolymers such as, by preference, vinyl acetate polymers, acrylic acid-acrylonitrile polymers, acrylic acid-acrylamide polymers;
d) ABA block copolymers, in which “A” denotes water-soluble groups or those swellable in water, such as by preference polyethylene oxide, polyvinyl alcohol, polyacrylamide, polyacrylic acid, polyvinylpyrrolidone, or polycaprolactone, and “B” denotes less-water-soluble or almost-water-insoluble groups such as, by preference, polypropylene oxide, polyvinyl acetate, polyvinyl butyral, polylauryl methacrylate, polystyrene, polyhydroxystearic acid, polysiloxanes,
e) AB graft (co)polymers, where “A” are water-soluble groups or those swellable in water, such as by preference vinyl alcohol, vinyl acetate, ethylene oxide, propylene oxide, vinylsulfonate, acrylic acids, and vinylamines, “B” is selected by preference from vinyl polymer or siloxane;
f) natural polymers, such as by preference cellulose derivatives, such as in particular carboxymethyl cellulose, hydroxypropylmethyl cellulose, methyl cellulose, and/or derivatives thereof.

Particularly to be mentioned in this context are polymers that by preference contain, at least in part, monomers that are selected from isobutyl methacrylate, n-butyl acrylate, n-butyl methacrylate, isobutyl acrylate, n-propyl acrylate, isopropyl methacrylate, methyl methacrylate, styrene, decyl (meth)acrylate, dodecyl (meth)acrylate, tetradecyl (meth)acrylate, and/or hexadecyl (meth)acrylate, and mixtures thereof.

According to a preferred embodiment, the particle contains an acidifying component, by preference carboxylic acid, advantageously polycarboxylic acids, in particular citric acid, adipic acid, succinic acid, glutaric acid, tartaric acid, sugar acids, aminocarboxylic acids, nitrilotriacetic acid (NTA), provided such use is not objectionable for environmental reasons, and/or mixtures thereof, by preference in the form of their sodium salts in each case.

According to a preferred embodiment, the particle contains at least one bleaching agent, selected by preference from the group encompassing perborates, in particular sodium perborate tetrahydrate and sodium perborate monohydrate, percarbonates, peroxypyrophosphates, citrate perhydrates, peracid salts and peracids, in particular perbenzoates, peroxophthalates, diperazelaic acid, phthaloimino peracid, or diperdodecanedioic acid.

According to a preferred embodiment, the particle contains at least one further substance usually contained in washing or cleaning agents, by preference a substance from the group of the surfactants, builder substances (inorganic and organic builder substances), bleaching agents, bleach activators, bleach stabilizers, bleach catalysts, enzymes, special polymers (for example those having co-builder properties), graying inhibitors, optical brighteners, UV protection substances, soil repellents, electrolytes, coloring agents, odorants, fragrances, perfume carriers, pH adjusting agents, complexing agents, fluorescing agents, foam inhibitors, wrinkle protection agents, antioxidants, quaternary ammonium compounds, antistatic agents, ironing adjuvants, UV absorbers, anti-redeposition agents, germicides, antimicrobial active substances, fungicides, viscosity regulators, luster agents, color transfer inhibitors, shrinkage preventers, corrosion inhibitors, preservatives, softeners, conditioners, protein hydrolysates, proofing and impregnating agents, hydrotropes, silicone oils, and swelling and anti-slip agents.

According to a preferred embodiment, the particle contains at least one substance selected from the following groups:
a) waxes such as carnauba, spermaceti, beeswax, lanolin, and/or corresponding derivatives;
b) hydrophobic plant extracts;
c) hydrocarbons such as squalene and/or squalane;
d) higher fatty acids, by preference having at least 12 carbon atoms, in particular lauric acid, myristic acid, palmitic acid, stearic acid, behenic acid, oleic acid, linoleic acid, linolenic acid, lanolinic acid, isostearic acid and polyunsaturated fatty acids;
e) higher fatty alcohols, by preference having at least 12 carbon atoms, in particular lauryl alcohol, cetyl alcohol, stearyl alcohol, oleyl alcohol, behenyl alcohol, cholesterol, and/or 2-hexadecanol;
f) esters such as cetyl octanoate, lauryl lactate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol distearate, glycerol tristearate, alkyl lactate, alkyl citrate, and/or alkyl tartrate;
g) lipids such as, for example, cholesterol, ceramides, and/or sucrose esters, and/or pseudo-ceramides;
h) vitamins such as, for example, vitamins A and E, vitamin alkyl esters such as e.g. vitamin C alkyl esters;
i) sun protection agents such as, for example, butyl methyoxybenzoylmethane;
j) phospholipids;
k) alpha-hydroxy acids and/or derivatives thereof;
l) germicides such as, for example, synthetic antimicrobial agents such as, for example 2-phenoxyethanol, and/or natural antimicrobial agents such as, for example, grapefruit extract or willow-bark extract;
m) mixtures of the aforesaid substances.

Advantageously, the aforesaid substances can be of benefit to the skin. Incorporation thereof into the particles according to the present invention is therefore advantageous in particular when the particles according to the present invention are intended to be the subject of a textile-care component. Care is therefore provided, for example, not only to the textile but also to the skin, since the aforesaid substances can be absorbed onto the textile fibers during textile treatment (e.g. textile laundering), and from there can be delivered onto the skin and then be of benefit thereto.

According to a further preferred embodiment of the invention, the particle is at least in part surrounded by a coating that by preference contains at least one component that is at least in part water-soluble or at least in part dispersible in water, which component is selected in particular from polyols, carbohydrates, starches, modified starches, starch hydrolysates, cellulose and cellulose derivatives, natural and synthetic gums, silicates, borates, phosphates, chitin and chitosan, water-soluble polymers, fat components, and mixtures thereof. Also suitable, for example, are waxes and/or resins, for example beeswax, benzoic resin, carnauba wax, candelilla wax, cumaron-indene wax, copals, shellac, mastic, polyethylene wax oxidates, or sandarac resin. Paraffins or gelatins, in particular including cellulose ethers, are also suitable.

According to a further preferred embodiment, the optional coating comprises polycarboxylates.

The optional coating of the particles can be performed in the manners described in the existing art. The optional coating material by preference surrounds the respective particle entirely, although a discontinuous coating can also be desirable. Appropriate potential coating materials are chiefly those that are commonly utilized in connection with washing or cleaning agents.

Materials that can be used as potential coating materials for purposes of the invention are any inorganic and/or organic substances and/or substance mixtures, by preference those that are sensitive to pH, temperature, and/or ionic strength, so that as a function of a change in pH, temperature, and/or ionic strength they lose their integrity, i.e., for example, entirely or partially dissolve.

Particularly preferred as coating materials are polymers and/or copolymers that have film-forming properties and can by preference be used from an aqueous dispersion. The critical magnitude for the film-forming properties is the glass transition temperature of the film-forming polymer and/or copolymer. Above the glass temperature, the polymer or copolymer is elastic, meltable, and flowable, whereas below the glass temperature it becomes brittle. Only above the glass transition temperature can the polymer easily be processed, as is necessary to form a film coating. The glass transition temperature can be influenced by the addition of low-molecular-weight substances having softening properties, the so-called plasticizers. In addition to the polymer, plasticizers can therefore also be used in the aqueous dispersion. Suitable as plasticizers are all substances that lower the glass transition temperature of the (by preference pH-sensitive) polymers and/or copolymers that are used. The polymer can thus be applied at lower temperatures, if applicable even at room temperature. Particularly preferred plasticizers are citric acid esters (by preference tributyl citrate and/or triethyl citrate), phthalic acid esters (by preference dimethyl phthalate, diethyl phthalate, and/or dibutyl phthalate), esters of organic polyalcohols (by preference glycerol triacetate), polyalcohols (by preference glycerol, propylene glycol), and/or polyoxyethylene glycols (by preference polyethylene glycol). The plasticizer becomes deposited between the polymer chains and thereby increases mobility, decreases interactions, and prevents friction and cracking of the film by decreasing brittleness.

It is particularly advantageous if the potential coating material contains a polyacrylate and/or a derivative thereof and/or a corresponding copolymer based on acrylic acid esters or acrylic acids and other monomers. Copolymers of acrylamide and acrylic acid, and or their derivatives, are especially advantageous for the potential coating material.

When the particle comprises, at least in part, a coating that encompasses a component that is water-soluble at least in part or water-dispersible at least in part, which component encompasses 0 wt % to 80 wt % of at least one solid polyol having by preference more than three hydroxyl radicals and 20 wt % to 100 wt % of a liquid diol or polyol in which the perfume is substantially insoluble and in which the solid polyol is substantially soluble, the aforesaid liquid polyol being selected by preference from glycerol, ethylene glycol, and diglycerol or mixtures thereof, and in which the solid polyol is by preference selected from glucose, sorbitol, maltose, glucamine, sucrose, polyvinyl alcohol, starch, alkylpolyglycoside, sorbitan fatty ester, polyhydroxy fatty acid amides whose fatty acid radicals contain 1 to 18 carbon atoms, and mixtures thereof, a further preferred embodiment of the invention then exists.

According to a preferred embodiment, the particle is colored. The particle can be through-colored, or its surface can be colored, or it can be coated with a colored substance.

Preferably, the particle can be colored by way of a coating. In a preferred embodiment, the coating comprises pigments, advantageously in the nanoscale region or micrometer region, by preference white pigments, in particular selected from titanium dioxide pigments such as, in particular, anatase pigments and/or rutile pigments, zinc sulfide pigments, zinc oxide (zinc white), antimony trioxide (antimony white), basic lead carbonate (lead white) 2PbCO₃.Pb(OH)₂, lithopone ZnS+BaSO₄. By preference, white adjuvants such as, by preference, calcium carbonate, talc 3MgO.4SiO₂.H₂O, and/or barium sulfate can also be contained.

In a further preferred embodiment, the pigments can be
a) colored pigments (by preference inorganic colored pigments, in particular iron oxide pigments, chromate pigments, iron blue pigments, chromium oxide pigments, ultramarine pigments, oxide mixed phase pigments, and/or bismuth vanadate pigments);
b) black pigments (e.g. aniline black, perylene black, iron oxide pigments, manganese black, and/or spinel black);
c) gloss pigments (by preference flaked effect pigments, metallic-effect pigments such as, e.g. aluminum pigments (silver bronze), copper pigments, and copper/zinc pigments (gold bronzes) and zinc pigments, luster pigments such as, for example, magnesium stearate, zinc stearate, lithium stearate, or ethylene glycol stearate or polyethylene terephthalate, interference pigments such as, for example, metal oxide/mica pigments); and/or
d) luminescent pigments such as, for example, azometbine fluorescent yellow, silver-doped and/or copper-doped zinc sulfide pigments.

If the particle size of the individual particles is substantially between 0.005 and 2.0 mm, this is then a more greatly preferred embodiment of the invention. The expression “substantially” means here that at least 40 wt %, advantageously at least 50 wt %, in more greatly advantageous fashion at least 60 wt %, in even more advantageous fashion at least 70 wt %, by preference at least 80 wt %, in particular 90 wt % of the particles meet this particle-size requirement.

If the particles according to the present invention are present in agglomerated fashion, the agglomerate size being by preference substantially 100 to 2000 μm, in particular substantially 100 to 800 μm, then once again a preferred embodiment of the invention exists. The expression “substantially” means here that at least 40 wt %, advantageously at least 50 wt %, in more greatly advantageous fashion at least 60 wt %, in even more advantageous fashion at least 70 wt %, by preference at least 80 wt %, in particular 90 wt % of the agglomerates exhibit the aforesaid agglomerate size. These agglomerated particles by preference disintegrate upon contact with water back into the smaller primary particles of which the agglomerates are/were made up. In some cases the agglomerate size can even be in the range from 0.1 to 30 mm if this is desired in terms of applications engineering.

The particle according to the present invention can have any shape. In a very particularly preferred embodiment, however, the particle is spherical (ball-shaped) or at least of approximately spherical or approximately ellipsoidal shape. The ellipsoid is similar to the sphere, but the longitudinal axis and transverse axis are different.

According to another embodiment, the particle is rather
a) cubically shaped or at least approximately cubically shaped, or
b) parallelepipedally shaped (e.g. cuboidal), or at least approximately parallepipedally shaped, or
c) lamellar in shape (flake-shaped and the like), or at least of approximately lamellar shape, or
d) needle-like or fiber-like in shape, or at least approximately of needle-like or fiber-like shape.

A further subject of the invention is the use of the particles according to the present invention as washing or cleaning agents or as an additive to washing or cleaning agents. According to the present invention, the term “washing or cleaning agents” also encompasses, for example, textile care agents such as, for example, conditioners.

A further subject of the invention is a method for manufacturing particles according to the present invention, encompassing
a) making available carrier materials, by preference based on aqueous suspensions and encompassing sulfate, carbonate, and layered silicate, that advantageously also encompass nonionic surfactant and, if applicable, further inorganic and organic constituents, the aqueous suspensions then being dried, then
b) if applicable, impregnating the carrier material with at least one nonionic surfactant, and
c) loading the carrier material with perfume by mixing perfume and (impregnated) carrier material and/or by spraying perfume onto the (impregnated) carrier material, and
   optionally, coating the perfumed carrier material.

According to method step a), the carrier material according to the present invention, by preference based on aqueous suspensions of inorganic and organic constituents that advantageously encompass nonionic surfactants, is made available, the aqueous suspensions then being dried. A carrier material that contains nonionic surfactant as manufactured is preferably used.

It is particularly preferred in this context if carbon dioxide is generated in the drying material upon drying of the aqueous suspension.

“Drying” means, in the broadest sense, any technical drying capability with which water and/or another solvent can be removed from the aqueous suspensions so thoroughly that at the completion of drying, particles, i.e. particulate solids, occur which form the desired carrier material. These particles of course need not be entirely solvent-free and/or anhydrous; for example, they can still contain considerable quantities of solvent and/or water, but by preference they have water concentrations below 30 wt %, advantageous below 25 wt %, in particular below 25%, based in each case on the solid occurring at the completion of drying. The water content can also be lower if desired, for example below 15 wt % or below 10 wt % or below 5 wt %, based in each case on the solid occurring at the completion of drying.

For drying, heat is advantageously delivered to the material to be dried. Drying can by preference take place in co-current, countercurrent, or crosscurrent fashion. Depending on the type of heat delivery, a distinction is made among, for example, contact driers, convection driers, and radiation driers. A subdivision can be made into, for example, overpressure, normal-pressure, and vacuum driers depending on the pressure existing in the drier. In convection drying, heat is transferred to the material to be dried predominantly by hot gases (air or inert gas), which is preferred. For this, for example, channel, chamber, belt, shaft, fluidized-bed, and/or atomization driers are used, which is preferred. In contact drying, which is likewise preferred, heat transfer takes place via heat exchanger surfaces. Among the contact driers are, for example, the roller, tube, and cabinet driers. Tray, plate, drum, and paddle driers operate according to both heat-delivery principles.

A drying method that is very preferred according to the present invention is spray drying. Fluidized-bed methods are also preferred for drying.

According to a preferred embodiment of the invention, the aqueous suspension to be dried contains substance(s) that release(s) carbon dioxide at elevated temperatures, selected by preference from hydrogencarbonate compounds, citric acid, and/or aconitic acid. Among the hydrogencarbonate compounds, sodium hydrogencarbonate is preferred.

According to a further preferred embodiment of the invention, the aqueous suspension to be dried according to the present invention contains 0 to 40 wt %, by preference 0.1 to 4 wt %, in particular 1 to 3 wt % citric acid, or 0 to 50 wt %, by preference 0.1 to 5 wt %, in particular 1 to 4 wt % hydrogencarbonate compound, or 0 to 40 wt %, by preference 0.1 to 10 wt %, in particular 1 to 5 wt % aconitic acid.

It may also be advantageous to use mixtures of hydrogencarbonate compounds, citric acid, and/or aconitic acid, such that the total quantity of such a mixture should not exceed 50 wt %, by preference 40 wt %, advantageously 20 wt %, but in particular 10 wt %, and such that a minimum total quantity should not fall below a value of 0.1 wt %, by preference 1 wt %, based in each case on the entire suspension.

It has been found, surprisingly, that in those cases in which carbon dioxide is released during drying, particles result that are notable for an even further improved ability to accept odorants.

If the carrier material that is made available still contains no nonionic surfactant after manufacture, then according to a preferred embodiment, impregnation of the carrier material according to the present invention with at least one nonionic surfactant then follows. Loading of the, by preference, nonionic-surfactant-containing carrier material with perfume then occurs, by mixing or spraying as just presented.

A further subject of the present invention is a detergent composition containing:
(A) particles according to the present invention,
(B) 0.01 wt % to 95 wt %, by preference 5 wt % to 85 wt %, advantageously 3 wt % to 30 wt %, in particular 5 wt % to 22 wt % additional surfactant(s),
(C) optionally, further ingredients of washing or cleaning agents.

In the context of this invention, the term “detergent composition” by preference means washing and/or cleaning agents and/or (textile) care agents and/or avivage agents and/or softener compositions.

In a preferred embodiment, the detergent composition according to the present invention is a multipotent washing or cleaning agent. These are agents having effects in multiple directions, for example two-in-one washing agents. These washing agents possess components having a washing or cleaning effect and components having a further effect, in particular a textile-care or avivage effect and/or a skin-care effect. “Skin-care effect” means here that the agent contains components that serve indirectly for skin care by the fact that during laundering they are transferred onto the textile, and they are then delivered onto the skin when the textiles are worn. Corresponding substances have already been recited above.

A three-in-one washing correspondingly combines components having a washing or cleaning effect and components having two further effects, in particular a textile-care or avivage effect and a skin-care effect.

If the additional surfactant encompasses anionic surfactant, by preference in a proportion of at least 50 wt % based on the entire quantity of additional surfactant, this is then a preferred embodiment, it being further preferred that the additional surfactant encompasses a mixture of anionic and nonionic surfactants.

A detergent composition according to the present invention that encompasses at least one surfactant, by preference at least two, from the group of the alkylbenzenesulfonates, alkyl estersulfonates, alkyl ethoxylates, alkylphenol alkoxylates, alkypolyglucosides, alkyl sulfates, alkyl ethoxysulfate, secondary alkyl sulfates, and/or mixtures thereof, these additional surfactants advantageously being contained in quantities from 1 wt % to 75 wt % based on the entire composition, represents a preferred embodiment.

Advantageous surfactants that can be contained in the detergent composition will be described below.

Anionic surfactants that can be used are, for example, those of the sulfonate and sulfate types. Possibilities as surfactants of the sulfonate type are, by preference, C₉₋₁₃ alkylbenzenesulfonates, olefinsulfonates, i.e. mixtures of alkene- and hydroxyalkanesulfonates, and disulfonates, for example such as those obtained from C₁₂₋₁₈ monoolefins having an end-located or internal double bond, by sulfonation with gaseous sulfur trioxide and subsequent alkaline or acid hydrolysis of the sulfonation products. Also suitable are alkanesulfonates that are obtained from C₁₂₋₁₈ alkanes, for example by sulfochlorination or sulfoxidation with subsequent hydrolysis and neutralization. The esters of α-sulfo fatty acids (estersulfonates), e.g. the α-sulfonated methyl esters of hydrogenated coconut, palm kernel, or tallow fatty acids, are likewise suitable.

Further suitable anionic surfactants are sulfonated fatty acid glycerol esters. “Fatty acid glycerol esters” are understood as the mono-, di- and triesters, and mixtures thereof, that are obtained during the production by esterification of a monoglycerol with 1 to 3 mol fatty acid, or upon transesterification of triglycerides with 0.3 to 2 mol glycerol. Preferred sulfonated fatty acid glycerol esters are the sulfonation products of saturated fatty acids having 6 to 22 carbon atoms, for example hexanoic acid, octanoic acid, decanoic acid, myristic acid, lauric acid, palmitic acid, stearic acid, or behenic acid.

Preferred alk(en)yl sulfates are the alkali, and in particular sodium, salts of the sulfuric acid semi-esters of the C₁₂-C₁₈ fatty alcohols, for example from coconut fatty alcohol, tallow fatty alcohol, lauryl, myristyl, cetyl, or stearyl alcohol, or the C₁₀-C₂₀ oxo alcohols, and those semi-esters of secondary alcohols of those chain lengths. Additionally preferred are alk(en)yl sulfates of the aforesaid chain length that contain a synthetic straight-chain alkyl radical produced on a petrochemical basis, which possess a breakdown behavior analogous to those appropriate compounds based on fat-chemistry raw materials. For purposes of washing technology, the C₁₂-C₁₆ alkyl sulfates and C₁₂-C₁₅ alkyl sulfates, as well as C₁₄-C₁₅ alkyl sulfates, are preferred. 2,3-alkyl sulfates that can be obtained, for example, as commercial products of the Shell Oil Company under the name DAN®, are also suitable anionic surfactants.

The sulfuric acid monoesters of straight-chain or branched C₇₋₂₁ alcohols ethoxylated with 1 to 6 mol ethylene oxide, such as 2-methyl-branched C₉₋₁₁ alcohols with an average of 3.5 mol ethylene oxide (EO) or C₁₂₋₁₈ fatty alcohols with 1 to 4 EO, are also suitable. Because of their high foaming characteristics they are used in cleaning agents only in relatively small quantities, for example in quantities from 1 to 5 wt %.

Other suitable anionic surfactants are also the salts of alkylsulfosuccinic acid, which are also referred to as sulfosuccinates or as sulfosuccinic acid esters and represent the monoesters and/or diesters of sulfosuccinic acid with alcohols, preferably fatty alcohols, and in particular ethoxylated fatty alcohols. Preferred sulfosuccinates contain C₈₋₁₈ fatty alcohol radicals or mixtures thereof. Particularly preferred sulfosuccinates contain a fatty alcohol radical that is derived from ethoxylated fatty alcohols which, considered per se, represent nonionic surfactants (see below for description). Sulfosuccinates whose fatty alcohol radicals derive from ethoxylated fatty alcohols having a restricted homolog distribution are, in turn, particularly preferred. It is likewise also possible to use alk(en)yl succinic acid having by preference 8 to 18 carbon atoms in the alk(en)yl chain, or salts thereof.

Further appropriate anionic surfactants are, in particular, soaps. Saturated fatty acid soaps, such as the salts of lauric acid, myristic acid, palmitic acid, stearic acid, hydrogenated erucic acid, and behenic acid, are suitable, as are soap mixtures derived in particular from natural fatty acids, e.g. coconut, palm kernel, or tallow fatty acids.

The anionic surfactants, including the soaps, can be present in the form of their sodium, potassium, or ammonium salts, and as soluble salts of organic bases, such as mono-, di-, or triethanolamine. The anionic surfactants are preferably present in the form of their sodium or potassium salts, in particular in the form of the sodium salts.

It is preferred to use as nonionic surfactants (additional surfactants) alkoxylated, advantageously ethoxylated, in particular primary alcohols having by preference 8 to 18 carbon atoms and an average of 1 to 12 mol ethylene oxide (EO) per mol of alcohol, in which the alcohol radical can be linear or preferably methyl-branched in the 2-position, or can contain mixed linear and methyl-branched radicals, such as those that are usually present in oxo alcohol radicals. Particularly preferred, however, are alcohol ethoxylates having linear radicals made up of alcohols of natural origin having 12 to 18 carbon atoms, e.g. from coconut, palm, tallow, or oleyl alcohol, and an average of 2 to 8 EO per mol of alcohol. The preferred ethoxylated alcohols include, for example, C₁₂₋₁₄ alcohols having 3 EO or 4 EO, C₉₋₁₁ alcohols having 7 EO, C₁₃₋₁₅ alcohols having 3 EO, 5 EO, 7 EO, or 8 EO, C₁₂₋₁₈ alcohols having 3 EO, 5 EO, or 7 EO, and mixtures thereof, such as mixtures of C₁₂₋₁₄ alcohol having 3 EO and C₁₂₋₁₈ alcohol having 5 EO. The degrees of ethoxylation indicated represent statistical averages that can be an integer or a fractional number for a specific product. Preferred alcohol ethoxylates exhibit a restricted distribution of homologs (narrow range ethoxylates, NRE). In addition to these nonionic surfactants, fatty alcohols having more than 12 EO can also be used. Examples of these are tallow fatty alcohol having 14 EO, 25 EO, 30 EO, or 40 EO.

Also usable as further nonionic surfactants (additional surfactants) are alkyl glycosides of the general formula RO(G)ₓ, in which R denotes a primary straight-chain or methyl-branched (in particular methyl-branched in the 2-position) aliphatic radical having 8 to 22, by preference 12 to 18 carbon atoms; and G is the symbol denoting a glycose unit having 5 or 6 carbon atoms, preferably glucose. The degree of oligomerization x, which indicates the distribution of monoglycosides and oligoglycosides, is any number between 1 and 10; preferably x is between 1.2 and 1.4.

A further class of nonionic surfactants (additional surfactants) used in preferred fashion, which are used either as the only nonionic surfactant or in combination with other nonionic surfactants, is alkoxylated, preferably ethoxylated or ethoxylated and propoxylated, fatty acid alkyl esters, preferably having 1 to 4 carbon atoms in the alkyl chain, in particular fatty acid methyl esters.

Nonionic surfactants of the amine oxide type, for example N-cocalkyl-N,N-dimethylamine oxide and N-tallowalkyl-N,N-dihydroxyethylamine oxide, and the fatty acid alkanolamides, can also be suitable. The quantity of these nonionic surfactants is preferably no more than that of the ethoxylated fatty alcohols, in particular no more than half thereof.

Further suitable additional surfactants are polyhydroxy fatty acid amides of the formula

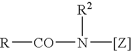

in which R denotes an aliphatic acyl radical having 6 to 22 carbon atoms; R² denotes hydrogen, an alkyl or hydroxyalkyl radical having 1 to 4 carbon atoms; and [Z] denotes a linear or branched polyhydroxyalkyl radical having 3 to 10 carbon atoms and 3 to 10 hydroxyl groups. The polyhydroxy fatty acid amides are known substances that can usually be obtained by reductive amination of a reducing sugar with ammonia, an alkylamine, or an alkanolamine, and subsequent acylation with a fatty acid, a fatty acid alkyl ester, or a fatty acid chloride.

Also belonging to the group of the polyhydroxy fatty acid amides are compounds of the formula

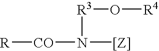

in which R denotes a linear or branched alkyl or alkenyl radical having 7 to 12 carbon atoms; R³ denotes a linear, branched, or cyclic alkyl radical or an aryl radical having 2 to 8 carbon atoms; and R⁴ denotes a linear, branched, or cyclic alkyl radical or an aryl radical or an oxyalkyl radical having 1 to 8 carbon atoms, C₁₋₄ alkyl or phenyl radicals being preferred; and [Z] denotes a linear polyhydroxyalkyl radical whose alkyl chain is substituted with at least two hydroxyl groups, or alkoxylated, preferably ethoxylated or propoxylated, derivatives of that radical.

[Z] is preferably obtained by reductive amination of a reducing sugar, for example glucose, fructose, maltose, lactose, galactose, mannose, or xylose. The N-alkoxy- or N-aryloxy-substituted compounds can then, for example, be converted into the desired polyhydroxy fatty acid amides by reaction with fatty acid methyl esters in the presence of an alkoxide as catalyst.

The detergent compositions according to the present invention, such as e.g. cleaning, care, and washing agents, can if applicable also contain cationic surfactants. Suitable cationic surfactants are, for example, also surface-active quaternary compounds, in particular having an ammonium, sulfonium, phosphonium, iodonium, or arsonium group, such as those that are also, for example, described in the existing art as antimicrobial active substances. The use of quaternary surface-active compounds having an antimicrobial effect allows the agent to be equipped with an antimicrobial effect, or allows its antimicrobial effect that may already be present on the basis of other ingredients to be improved.

Particularly preferred cationic surfactants are the quaternary ammonium compounds (QACs), in part having antimicrobial action, according to the general formula (R^{I})(R^{II})(R^{III})(R^{IV}) N⁺X⁻, in which R^{I} to R^{IV} represent identical or different C₁-C₂₂ alkyl radicals, C₇-C₂₈ aralkyl radicals, or heterocyclic radicals, two or (in the case of an aromatic bond such as in pyridine) even three radicals forming the heterocycle together with the nitrogen atom, for example a pyridinium or imidazolinium compound; and X⁻ are halide ions, sulfate ions, hydroxide ions, or similar anions. For an optimum antimicrobial action, at least one of the radicals preferably has a chain length from 8 to 18, in particular 12 to 16, carbon atoms.

QACs can be produced by the reaction of tertiary amines with alkylating agents such as, for example, methyl chloride, benzyl chloride, dimethyl sulfate, dodecyl bromide, but also ethylene oxide. The alkylation of tertiary amines having a long alkyl radical and two methyl groups is particularly easy; in addition, the quaternization of tertiary amines having two long radicals and one methyl group can also be carried out using methyl chloride under mild conditions. Amines that possess three long alkyl radicals or hydroxy-substituted alkyl radicals have little reactivity, and are preferably quaternized using dimethyl sulfate.

Suitable QACs are, for example, benzalkonium chloride (N-alkyl-N,N-dimethylbenzylammonium chloride, CAS No. 8001-54-5), benzalkon B (m,p-dichlorobenzyldimethyl-C₁₂-alkylammonium chloride, CAS No. 58390-78-6), benzoxonium chloride (benzyldodecyl-bis-(2-hydroxyethyl)ammonium chloride), cetrimonium bromide (N-hexadecyl-N,N-trimethylammonium bromide, CAS No. 57-09-0), benzetonium chloride (N,N-dimethyl-N-[2-[2-[p-(1,1,3,3-tetramethylbutyl)phenoxy]ethoxy]ethyl]benzylammonium chloride, CAS No. 121-54-0), dialkyldimethylammonium chlorides such as di-n-decyldimethylammonium chloride (CAS No. 7173-51-5-5), didecyldimethylammonium bromide (CAS No. 2390-68-3), dioctyldimethylammonium chloride, 1-cetylpyridinium chloride (CAS No. 123-03-5), and thiazoline iodide (CAS No. 15764-48-1), as well as mixtures thereof. Preferred QACs are the benzalkonium chlorides having C₈-C₁₈ alkyl radicals, in particular C₁₂-C₁₄ alkylbenzyldimethylammonium chloride. A particularly preferred QAC is cocopentaethyoxymethylammonium methosulfate (INCI: PEG-5 Cocomonium Methosulfate; Rewoquat® CPEM).

To avoid possible incompatibilities between the antimicrobial cationic surfactants and the anionic surfactants contained in the detergent composition according to the present invention, an anionic surfactant-compatible surfactant and/or one that is minimally cationic is used; or in a particular embodiment of the invention, antimicrobially active cationic surfactants are entirely omitted.

In place thereof, for example, parabens, benzoic acid and/or benzoate, lactic acid, salicylic acid, and/or lactates can be used as antimicrobially effective substances. Benzoic acid and/or lactic acid are particularly preferred.

The detergent compositions according to the present invention, such as cleaning, care, and washing agents, can contain one or more cationic surfactants in quantities, based on the entire composition, from 0 to 5 wt %, greater than 0 to 5 wt %, by preference 0.01 to 3 wt %, in particular 0.1 to 1 wt %.

The detergent compositions according to the present invention, such as cleaning, care, and washing agents, can also contain amphoteric surfactants. Suitable amphoteric surfactants are, for example, betaines of the formula (R¹)(R²)(R³)N⁺CH₂COO⁻, in which R¹ denotes an alkyl radical, interrupted if applicable by heteroatoms or heteroatom groups, having 8 to 25, by preference 10 to 21 carbon atoms, and R² and R³ denote identical or different alkyl radicals having 1 to 3 carbon atoms, in particular C₁₀-C₂₂ alkyldimethylcarboxylmethyl betaine and C₁₁-C₁₇ alkylamidopropyldimethylcarboxymethyl betaine. Also conceivable is the use of alkylamidoalkylamines, alkyl-substituted amino acids, acylated amino acids, or biosurfactants as amphoteric surfactants in the agents according to the present invention, such as cleaning, care, and washing agents.

The detergent compositions according to the present invention, such as cleaning, care, and washing agents, can contain one or more amphoteric surfactants in quantities, based on the total composition, from 0 to 5 wt %, greater than 0 to 5 wt %, by preference 0.01 to 3 wt %, in particular 0.1 to 1 wt %.

In addition to the substances having washing activity, detergency builders are the most important ingredients of washing or cleaning agents, i.e. in particular zeolites, silicates, carbonates, organic co-builders and even (where no environmental prejudices against their use exist) the phosphates. The detergent compositions can accordingly preferably also contain additional detergency builders alongside the detergency builders present in the particles according to the present invention. If the detergent composition therefore furthermore encompasses at least 1 wt % of an additional washing agent detergency builder, a further preferred embodiment of the invention then exists, it being likewise preferred if usual additional constituents for washing or cleaning agents are also contained.

Those additional detergency builders already recited are, for example, advantageous. Crystalline, layered-form sodium silicates or amorphous sodium silicates are preferably suitable. Zeolite, by preference zeolite A and/or zeolite P, is likewise advantageous. Zeolite MAP® (commercial product of the Crosfield Co.) is particularly preferred as zeolite P. Also suitable, however, are zeolite X as well as mixtures of A, X, and/or P. Also commercially available and preferred for use in the context of the present invention is, for example, a co-crystal of zeolite X and zeolite A (approx. 80 wt % zeolite X) that is marketed by CONDEA Augusta S.p.A. under the trade name VEGOBOND AX® and can be described by the formula

nNa₂O.(1-n)K₂O.Al₂O₃.(2-2.5)SiO₂ (3.5-5.5)H₂O.

The zeolite can be used both as a further detergency builder and as a dusting on the particles. Suitable zeolites exhibit by preference an average particle size of less than 10 μm (volume distribution; measurement method: Coulter Counter), and by preference contain 18 to 22 wt %, in particular 20 to 22 wt %, bound water.

The use of other usual additional detergency builders is of course also advantageous, for example the commonly known phosphates are possible as builder substances in the detergent composition provided such use is not to be avoided for environmental reasons. The sodium salts of the orthophosphates, pyrophosphates, and in particular tripolyphosphates are particularly suitable.

Usable organic builder substances are, for example, the polycarboxylic acids usable in the form of their sodium salts, such as citric acid, adipic acid, succinic acid, glutaric acid, tartaric acid, sugar acids, aminocarboxylic acids, nitrilotriacetic acid (NTA), provided such use is not objectionable for environmental reasons, as well as mixtures thereof. Preferred salts are the salts of the polycarboxylic acids such as citric acid, adipic acid, succinic acid, glutaric acid, tartaric acid, sugar acids, and mixtures thereof.

In addition to the constituents already recited, further ingredients usual in washing or cleaning agents, in particular from the group of the bleaching agents, bleach activators, enzymes, enzyme stabilizers, fluorescing agents, dyes, foam inhibitors, silicone oils, anti-redeposition agents, optical brighteners, graying inhibitors, color transfer inhibitors, and corrosion inhibitors can be introduced into the detergent composition or be contained therein. Further ingredients to be optionally used derive, for example, from the group of the oligomeric and polymeric polycarboxylates, pH adjusting agents, shrinkage preventers, wetting improvers, antimicrobial active substances, germicides, fungicides, antioxidants, antistatic agents, ironing adjuvants, proofing and impregnating agents, swelling and anti-slip agents, chelating agents, textile softeners, and UV absorbers.

Among the compounds yielding H₂O₂ in water that serve as bleaching agents, sodium perborate tetrahydrate and sodium perborate monohydrate have particular importance. Additional usable bleaching agents are, for example, sodium percarbonate, peroxypyrophosphates, citrate perhydrates, and peracid salts or peracids that yield H₂O₂, such as perbenzoates, peroxophthalates, diperazelaic acid, phthaloimino peracid, or diperdodecanedioic acid.

To achieve an improved bleaching effect when washing at temperatures of 60° C. and below, bleach activators can be incorporated. Compounds that, under perhydrolysis conditions, yield aliphatic peroxycarboxylic acids having by preference 1 to 10 carbon atoms, in particular 2 to 4 carbon atoms, and/or optionally substituted perbenzoic acid, can be used as bleach activators. Substances that carry O- and/or N-acyl groups having the aforesaid number of carbon atoms, and/or that carry optionally substituted benzoyl groups, are suitable. Multiply acylated alkylenediamines, in particular tetraacetylethylenediamine (TAED), acylated triazine derivatives, in particular 1,5-diacetyl-2,4-dioxohexahydro-1,3,5-triazine (DADHT), acylated glycolurils, in particular tetraacetyl glycoluril (TAGU), N-acylimides, in particular N-nonanoyl succinimide (NOSI), acylated phenolsulfonates, in particular n-nonanoyl- or isononanoyloxybenzenesulfonate (n- and iso-NOBS), carboxylic acid anhydrides, in particular phthalic acid anhydride, acylated polyvalent alcohols, in particular triacetin, ethylene glycol diacetate, and 2,5-diacetoxy-2,5-dihydrofuran, are preferred.

In addition to or instead of the conventional bleach activators, so-called bleach catalysts can also be incorporated. These substances are bleach-enhancing transition-metal salts or transition-metal complexes such as, for example, Mn, Fe, Co, Ru, or Mo salt complexes or carbonyl complexes. Mn, Fe, Co, Ru, Mo, Ti, V, and Cu complexes having nitrogen-containing tripod ligands, as well as Co, Fe, Cu, and Ru ammine complexes, are also applicable as bleach catalysts.

Suitable enzymes are those of the protease, lipase, amylase, and cellulase classes, and mixtures thereof. Enzymatic active substances obtained from bacterial strains or fungi, such as Bacillus subtilis, Bacillus licheniformis, and Streptomyceus griseus, are particularly suitable. Proteases of the subtilisin type, and in particular proteases obtained from Bacillus lentus, are preferably used. Enzyme mixtures, for example of protease and amylase or protease and lipase or protease and cellulase, or of cellulase and lipase, or of protease, amylase, and lipase or protease, lipase and cellulase, but in particular cellulase-containing mixtures, are of particular interest in this context. Peroxidases or oxidases have also proven suitable in certain cases. The enzymes can be adsorbed onto carrier substances and/or embedded in enveloping substances in order to protect them from premature breakdown. The proportion of enzymes, enzyme mixtures, or enzyme granulates in the compositions according to the present invention can be, for example, approximately 0.1 to 5 wt %, by preference 0.1 to approximately 2 wt %. Preferred embodiments are entirely enzyme-free, i.e. contain 0 wt % enzymes.

The compositions can also contain components (so-called soil repellents) that positively influence the ability of oils and fats to be washed out of textiles. This effect becomes particularly apparent when the soiled textile is one that has already been previously washed several times with a washing or cleaning agent according to the present invention that contains this oil- and fat-dissolving component. The preferred oil- and fat-dissolving components include, for example, nonionic cellulose ethers such as methyl cellulose and methylhydroxypropyl cellulose having a 15 to 30 wt % proportion of methoxy groups and a 1 to 15 wt % proportion of hydroxypropoxyl groups, based in each case on the nonionic cellulose ethers, as well as polymers, known from the existing art, of phthalic acid and/or terephthalic acid and of their derivatives, in particular polymers of ethylene terephthalates and/or polyethylene glycol terephthalates or anionically and/or nonionically modified derivatives thereof. Of these, the sulfonated derivates of phthalic acid polymers and terephthalic acid polymers are particularly preferred.

The compositions can contain, as optical brighteners, derivatives of diaminostilbenedisulfonic acid or its alkali metal salts. Suitable, for example, are salts of 4,4′-bis(2-anilino-4-morpholino-1,3,5-triazinyl-6-amino)stilbene-2,2′-disulfonic acid, or compounds of similar structure that carry, instead of the morpholino group, a diethanolamino group, a methylamino group, an anilino group, or a 2-methoxyethylamino group. Brighteners of the substituted diphenylstyryl type can also be present, e.g. the alkali salts of 4,4′-bis(2-sulfostyryl)diphenyl, of 4,4′-bis(4-chloro-3-sulfostyryl)diphenyl, or of 4-(4-chlorostyryl)-4′-(2-sulfostyryl)diphenyl. Mixtures of the aforesaid brighteners can also be used.

In order to improve the aesthetic impression of the agents according to the present invention, it can be colored (by preference, in part) with suitable dyes. Preferred dyes, the selection of which will present no difficulty whatsoever to one skilled in the art, possess excellent shelf stability and insensitivity to the other ingredients of the agents and to light, and no pronounced substantivity with respect to textile fibers, in order not to color them.

If the detergent composition according to the present invention is present in the form of agglomerates, this is then likewise a preferred embodiment of the invention.

If the detergent composition according to the present invention has a density of, advantageously, at least 300 g/l, advantageously 400 g/l, in more advantageous fashion 500 g/l, by preference at least 600 g/l, and in particular at least 650 g/l, this is then likewise a preferred embodiment of the invention.

If the detergent composition according to the present invention further encompasses a second perfume that is sprayed onto the surface of the detergent grains that are contained, then a preferred embodiment of the invention once again exists.

A further preferred embodiment is a detergent composition according to the present invention in the form of a laundry washing-agent element, by preference in tablet form.

For example, the particles according to the present invention that are contained in the washing-agent element can also contain disintegration accelerators, for example substances that possess a great ability to absorb water (e.g. starch, cellulose derivatives, alginates, dextrans, crosslinked polyvinylpyrrolidone, casein derivatives, etc.) and/or, in particular, gas-evolving substances (e.g. sodium hydrogencarbonate and citric acid or tartaric acid, etc.), so that an effervescent effect or bubbling effect occurs.

A further preferred embodiment is a detergent composition according to the present invention that is decanted into pouches, bags, or sacklets.

The pouches, bags, or sacklets are by preference configured so that they permit penetration of the detergent composition in the course of the washing operation. They thus either are water-permeable or dissolve in water, if applicable under adjustable conditions (temperature, pH, ionic strength).

A further subject of the present invention is a method for washing textiles, encompassing the step of bringing the textiles into contact with an aqueous medium that contains an effective quantity of a detergent composition that encompasses particles according to the present invention.

A further subject of the present invention is a washing- or cleaning-agent additive that encompasses particles according to the present invention, this additive being present in the form of a bag or pouch, the quantity measured into the bag or pouch being such that it suffices for one normal wash load of an automatic washing machine.

A further subject of the invention is a textile conditioning composition that is added during the rinse cycle of an automatic washing machine, encompassing particles according to the present invention.

As used herein, and in particular as used herein to define the elements of the claims that follow, the articles “a” and “an” are synonymous and used interchangeably with “at least one” or “one or more,” disclosing or encompassing both the singular and the plural, unless specifically defined otherwise. The conjunction “or” is used herein in both in the conjunctive and disjunctive sense, such that phrases or terms conjoined by “or” disclose or encompass each phrase or term alone as well as any combination so conjoined, unless specifically defined otherwise.

The description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed.

Practical and preferred embodiments of the invention can be further illustrated by means of the following examples, which are not intended as limiting the invention. Changes in form and substitution of equivalents are contemplated as circumstances may suggest or render expedient. Although specific terms have been employed herein, such terms are intended in a descriptive sense and not for purposes of limitation.

### EXAMPLE

The particles according to V1 and V2, as well as E1, were each manufactured using a standard spray-drying method.

| | V1 | V2 | E1 |
|---|---|---|---|
| Bentonite | — | — | 10.5 |
| C₁₂-C₁₈ fatty alcohol + 4.5 EO | 1.45 | 1.4 | — |
| C₁₂-C₁₈ fatty alcohol + 7 EO | 0.5 | — | — |
| Zeolite A (anhydrous active substance) | 76.4 | — | — |
| Sodium silicate 2.0 | — | 1.0 | 9.56 |
| Sodium sulfate | 1.7 | 1.0 | 50.85 |
| Carboxymethyl cellulose sodium salt | 2.0 | 2.0 | 1.02 |
| Sodium carbonate | — | 83.4 | 19.58 |
| Polyacrylate | — | — | 3.75 |
| Water | 16.75 | 10.00 | 4.36 |
| other | 1.2 | 1.2 | 0.38 |
| Total | 100.00 | 100.00 | 100.00 |
| Scent stability** after 4 weeks of storage | poor | moderate | good |
| Perfume oil acceptance capacity* | 16 wt % | <10 wt % | 20 wt % |

*Perfume oil acceptance capacity: The particles according to the present invention in accordance with E1 were able to accept the largest quantities of perfume oil, namely 20 wt %, which means that 100 g of the particles had, after perfume impingement, a weight of 120 g. They retained their good powder properties, i.e. remained easy to pour and exhibited no clumps.
**Scent stability: The scent stability was evaluated by a six-member panel of persons with no perfume-technology experience. They assessed the scent of the particles according to V1 and V2, and E1, approximately 24 hours after the particles had been loaded with the quantity of an odorant composition*** (perfume oil) reproduced in the table.

They also assessed the scent of these particles after four weeks of storage. It was found consistently that the scent of the particles according to VI had changed in distinctly disadvantageous fashion after four weeks of storage. The scent contained unpleasant notes that had not been present before storage. The original pleasant odor had been very much impaired after this storage period. The scent of the particles according to V2 was judged to be better compared to VI; the unpleasant notes were less intense than with the particles according to VI, but were nevertheless intrusively perceptible.

In contrast thereto, the scent of the particles according to E1 was still good even after four weeks of storage; the pleasant odor was retained and had changed, if at all, only gradually. The six-member panel therefore judged scent stability after four weeks of storage to be “poor” for the particles according to V1, and “moderate” for those according to V2. The corresponding scent stability of the particles according to E1, on the other hand, was judged to be “good.”

***Odorant composition: The odorant composition that was used contained, in addition to other components, 15 wt % allyl amyl glycolate and 15 wt % cyclogalbanate, based in each case on the entire odorant composition.

1. A particle comprising a carbonate, a sulfate, a perfume, and a layered silicate, the particle having a weight ratio of layered silicate to carbonate and sulfate combined of ≦1:2.

2. The particle of claim 1, wherein the layered silicate comprises a clay.

3. The particle of claim 2, wherein the clay comprises bentonite.

4. The particle of claim 1, comprising a nonionic surfactant.

5. The particle of claim 4, comprising at least 0.1% by weight of the nonionic surfactant.

6. The particle of claim 1, comprising a polymeric clay flocculent.

7. The particle of claim 6, wherein the clay flocculent comprises a polymer or copolymer of one or more monomers selected from the group consisting of ethylene oxide, acrylamide, acrylic acid, dimethyl aminoethyl methacrylate, vinyl alcohol, vinylpyrrolidone, and ethyleneimine.

8. The particle of claim 6, comprising 0.005% to 20% by weight of the polymeric clay flocculent, based on the weight of the layered silicate.

9. The particle of claim 1, comprising one or more compounds selected from the group consisting of cationic surfactants, zwitterionic compounds, ampholytes, amphosurfactants, betaines, cationic polymers, and amphoteric polymers.

10. The particle of claim 1, comprising a quaternary ammonium compound.

11. The particle of claim 10, comprising 0.1% to 30% by weight of the quaternary ammonium compound, based on the entire particle weight.

12. The particle of claim 1, comprising 0.1% to 30% by weight of an organic humectant, based on the entire particle weight.

13. The particle of claim 12, wherein the organic humectant comprises one or more compounds selected from the group consisting of glycerol, ethylene glycol, propylene glycol, glycerol dimers, and glycerol trimers.

14. The particle of claim 1, comprising a complexing agent.

15. The particle of claim 1, comprising pentaerythrite or a derivative thereof.

16. The particle of claim 1, comprising an alkali silicate.

17. The particle of claim 1, comprising an acidifying component.

18. The particle of claim 1, comprising one or more components selected from the group consisting of surfactants, builder substances, bleaching agents, bleach activators, bleach stabilizers, bleach catalysts, enzymes, polymers, graying inhibitors, optical brighteners, UV protection substances, soil repellents, electrolytes, coloring agents, odorants, perfume carriers, pH adjusting agents, complexing agents, fluorescing agents, foam inhibitors, wrinkle protection agents, antioxidants, quaternary ammonium compounds, antistatic agents, ironing adjuvants, UV absorbers, anti-redeposition agents, germicides, antimicrobial active substances, fungicides, viscosity regulators, luster agents, color transfer inhibitors, shrinkage preventers, corrosion inhibitors, preservatives, softeners, conditioners, protein hydrolysates, proofing and impregnating agents, hydrotropes, silicone oils, and swelling and anti-slip agents.

19. The particle of claim 1, comprising a coating.

20. A method of manufacturing a perfumed particle, comprising the steps of:
a) preparing an aqueous suspension of carrier materials, said suspension comprising a sulfate, a carbonate, and a layered silicate;
b) drying the suspension to form a particle comprising the carbonate, the sulfate, and the layered silicate, the particle having a weight ratio of layered silicate to carbonate and sulfate combined of ≦1:2;
c) optionally impregnating the particle with a nonionic surfactant;
d) loading the particle with perfume by mixing perfume with or spraying perfume onto the particle; and
e) optionally, coating the perfumed particle.

21. A detergent, comprising the particle of claim 1 and 0.01% to 95% by weight of one or more surfactants.

22. The detergent of claim 21, comprising 3% to 30% by weight of the one or more surfactants.

23. A method of washing textiles, comprising contacting a textile with an aqueous medium containing a quantity of the detergent of claim 21 effective to wash said textile.

24. A washing- or cleaning-agent additive, comprising the particle of claim 1, said additive being in the form of a pouch.

25. A textile softener composition, comprising a textile softening compound and the particle of claim 1.
